(19) United States
(12) Patent Application Publication
(10) Pub. No.: US 2003/0104974 A1
(43) Pub. Date: Jun. 5, 2003

(54) Dual inhibitorsof PDE 7 and PDE 4

(76) Inventors: William J. Pitts, Newtown, PA (US); Andrew J. Watson, West Windsor, NJ (US); John H. Dodd, Stockton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/135,998

(22) Filed: Apr. 30, 2002

Related U.S. Application Data
(60) Provisional application No. 60/287,964, filed on May 1, 2001.
(60) Provisional application No. 60/299,287, filed on Jun. 19, 2001.
(60) Provisional application No. 60/368,752, filed on Mar. 29, 2002.

(51) Int. Cl. A61K 31/00

(52) U.S. Cl. 514/1

(57) ABSTRACT

Dual inhibitors of PDE7 and PDE4, together with their use to treat leukocyte activation-associated disorders (including transplant rejection, rheumatoid arthritis, inflammatory bowel disease, psoriasis, asthma, chronic obstructive pulmonary disease, lupus and multiple sclerosis), are provided herein.

This application claims priority to U.S. Provisional Application 60/287,964 filed May 1, 2001, U.S. Provisional Application 60/299,287 filed Jun. 19, 2001, and U.S. Provisional Application 60/368,752 filed Mar. 29, 2002. The entirety of each of these applications is incorporated herein by reference.

### FIELD OF THE INVENTION

The present invention relates to dual inhibitors of phosphodiesterase 7 (PDE 7) and phosphodiesterase 4 (PDE 4), pharmaceutical compositions containing these inhibitors, and the use of these inhibitors in the treatment of leukocyte activation-associated or leukocyte-activation mediated disease and inflammatory diseases. The present invention further provides for a method of reducing or alleviating nausea and emesis associated with the administration of PDE4 inhibitors comprising either the administration of a dual PDE7-PDE4 inhibitor, or the simultaneous or sequential co-administration of a selective PDE 7 inhibitor together with a selective PDE 4 inhibitor.

### BACKGROUND OF THE INVENTION

Phosphodiesterases (PDES) hydrolyze the second messenger molecules cAMP and cGMP to affect cellular signaling. At least 11 families of PDEs exist, some of which (PDE3,4,7,8) are specific for cAMP, and others (PDE5,6,9) for cGMP. Further family members (PDE1,2,10,11) have dual specificity. A recent publication demonstrated a role for PDE7 in the activation and/or proliferation of T cells(Li, Yee and Beavo, Science 283:848-851, 1999). Resting T lymphocytes express mainly PDE3 and PDE4. However, upon activation, T cells dramatically upregulate PDE7 and appear to rely on this isozyme for regulation of cAMP levels. Removal of the ability to upregulate the production of PDE7 protein by anti-sense oligonucleotides inhibited the proliferation and IL-2 production along with the maintenance of high concentrations of intracellular cAMP in CD3×CD28 stimulated T cells. Inhibition of PDE4 has been associated with an antiinflammatory response associated with other leukocytes such as monocytes, macrophages, mast cells, basophils and neutrophils. The combined activity of the present dual PDE7/4 inhibitors on leukocyte activation may be especially useful in treating a wide variety of immune and inflammatory disorders.

Several isoforms of PDE1 have been identified and are distributed in heart, lung, and kidney tissue, as well as in circulating blood cells and smooth muscle cells. PDE1 inhibitors have demonstrated potent vasodilator activity. Such activity might produce an undesirable side effect in a therapeutic agent with the utilities provided herein for a dual PDE7-PDE4 inhibitor.

The PDE3 family of enzymes are distributed in several tissues including the heart liver, and platelets. PDE3 inhibitors have demonstrated potent cardiac inotropic activity. Such activity would represent an undesirable side effect in a therapeutic agent with the utilities provided herein for a dual PDE7-PDE4 inhibitor.

PDE5 inhibitors (for example sildenafil) have been used clinically for the treatment of erectile dysfunction, due to expression of PDE5 in the human corpus cavernosum smooth muscle. Inhibition of PDE5, however, does not cause a significant incidence of erection in the absence of sexual stimulation. Inhibition of PDE6 has been associated with visual disturbances consisting of altered color perception.

The function of other PDE family members, such as PDE8, PDE9, PDE10, and PDE11, is not clear at the present time. A recent publication suggests that PDE8A1 is also up-regulated in activated T cells, although no functional significance of this observation has been demonstrated (Glavas, Ostenson, Schaefer, Vasta and Beavo, PNAS 98(11): 6319-6324, 2001).

Several isoforms of PDE4 exist, and these are expressed in a wide variety of tissues including heart, kidney, brain, the gastrointestinal track and circulating blood cells. PDE4 inhibitors have demonstrated clinical utility for COPD, and have also been suggested to have utility for the various forms of asthma, rheumatoid arthritis, and multiple sclerosis, and to possess anti-inflammatory activity.

There has been an abundance of research directed at discovery and therapeutic applications of PDEA inhibitors (Dyke, and Montana, Expert Opin. Investig. Drugs 11(1): 1-13, 2002). Cilomilast (ARIFLO) is a selective, prototypical PDE4 inhibitor which has been in clinical trials for the treatment of asthma and COPD. At present nausea and emesis remain the major obstacles in the development of PDE4 inhibitors. (Huang, Ducharme, Macdonald, and Robichard, Current Opin. Chem. Bio. 5, 432-438, 2001) Two approaches to minimize the dose limiting nausea and emesis of PDE4 inhibitors include: (1) selection of PDE4 inhibitors which have decreased binding to the high affinity rolipram binding site, and (2) selectivity for a specific PDE4 subtype.

We have discovered that co-administration of a selective PDE7 inhibitor with a selective PDE4 inhibitor, or use of a dual PDE7-PDE4 inhibitor, would result in increased therapeutic effectiveness over the prior approaches. This increase in efficacy would result in an increase in the therapeutic window with regard to nausea and emesis, and represent a significant improvement over the administration of a PDE4 inhibitor as a single agent. Co-administration of a selective PDE4 inhibitor with a selective PDE7 inhibitor is expected to have a similar activity to a dual PDE7-PDE4 as discussed below.

Co-administration of a selective PDE4 inhibitor and a selective PDE7 inhibitor, or the administration of a dual PDE7-PDE4 inhibitor, is expected to have broad application as an immunosuppressant therapy in leukocyte activation-associated or leukocyte-activation mediated disease. PDE7 inhibitors will act at a different stage of the T cell signaling process compared to current immunosuppressants by inhibiting a very early stage of the T cell activation cascade, as a result of its PDE7 inhibitory activity. A dual PDE7-PDE4 inhibitor, as a result of its PDE4 inhibition, is also expected to have application to a number of allergic and inflammatory diseases. This results in part due to an ability of PDE4 inhibitors to decrease the production of the pro-inflammatory cytokines such as Tumor Necrosis Factor alpha, (TNF-α) in monocytes and macrophages, as well as affect granulocytes such as neutrophils etc. Thus, dual PDE4I7 inhibitors would be expected to be particularly useful in treating disorders that (1) are alleviated at least in part by PDE7 inhibition (e.g., though decreased T cell activation), and (2) involve one or more inflammatory response alleviated by at least in part by PDE4 inhibition (e.g., via decreased mast cell, basophil and neutrophil degranulation and monocyte and macrophage production of pro-inflammatory cytokines such as TNF-alpha). A dual PDE7-PDE4 inhibitor is also expected to have a decreased potential for clinically significant side effects compared to current immunosuppressants. As such dual PDE7-PDE4 inhibitors would be particularly useful in treatment of disorders such as solid organ transplantation (SOT) and rheumatoid arthritis, inflammatory bowel disease (IBD), psoriasis, asthma, chronic obstructive pulmonary disease (COPD), lupus and multiple sclerosis.

Development of dual PDE7-PDE4 inhibitors will yield novel classes of therapeutics and have a novel mechanism of action by maintaining high levels of intracellular cAMP. These inhibitors would target a major unmet medical need in an area where current therapies possess significant toxicity.

Two PDE7 genes (PDE7A and PDE7B) have been identified. PDE7A (EC 3.1.4.17) has three isoforms generated by alternate splicing; PDE7A1 restricted mainly to T cells and the brain, PDE7A2 for which mRNA is expressed in a number of cell types including muscle cells, and PDE7A3 found in activated T cells. The PDE7A1 and PDE7A2 isoforms have different sequence at the amino termini, and it is thought that this portion of each molecule is likely to be important for cellular localization of the enzyme. However, the catalytic domain of each PDE7A enzyme is identical (Han, P., Zhu, X. and Michaeli, T. Alternative splicing of the high affinity cAMP-specific phosphodiesterase (PDE7A) mRNA in human skeletal muscle and heart. J. Biol. Chem. 272 (26), 16152-16157 (1997)). Although abundant PDE7A2 mRNA has been identified, the presence of active enzyme in tissues is controversial, as no convincing data shows PDE7A2 protein in situ in the adult. PDE7A3 is similar to PDE7A1 in the amino terminus but has a different carboxy terminal sequence than PDE7A1 and PDE7A2. The enzymatic activity for PDE7A3 has not been characterized.

PDE7B (EC 3.1.4.17), a second PDE7 gene family member, has approximately 70% homology to PDE7A in the enzymatic core (Sasaki, T., Kotera, J., Yuasa, K and Omori, K Identification of human PDE7B, a cAMP-specific phosphodiesterase Biochem. Biophys. Res. Commun. 271 (3), 575-583 (2000)).

### SUMMARY OF THE INVENTION

The present invention provides for novel heterocyclic compounds that are dual inhibitors of PDE7 and PDE4. Additionally, the present invention provides for the use of dual PDE7/PDE4 inhibitors to treat leukocyte activation-associated or leukocyte activation-mediated diseases and inflammatory diseases. Additionally this invention provides for the simultaneous or sequential co-administration of a selective PDE4 inhibitor with a selective PDE7 inhibitor.

Dual inhibitor compounds within the scope of the present invention include compounds of Formula Ia and Ib, pharmaceutically acceptable salts, prodrugs and solvates thereof:

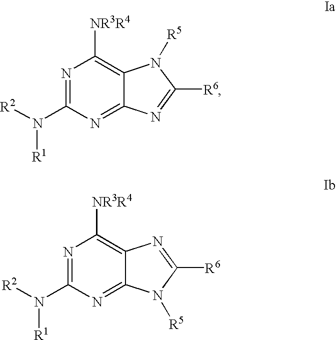

wherein

R¹ is H or alkyl;

R¹ is optionally substituted heteroaryl, or 4-substituted aryl;

R³ is hydrogen or alkyl;

R⁴ is alkyl, optionally substituted (aryl)alkyl, optionally substituted (heteroaryl)alkyl, optionally substituted heterocylo, or optionally substituted (heterocyclo)alkyl;

or R³ and R⁴ together with the nitrogen atom to which they are attached may combine to form an optionally substituted heterocyclo ring;

R⁵ is alkyl, optionally substituted (aryl)alkyl, or optionally substituted (heteroaryl)alkyl; and

R⁶ is hydrogen or alkyl.

Preferred compounds within Formula Ia and Ib are those wherein

R¹ is hydrogen

R² is thiazolyl, oxazolyl, or isoxozolyl (preferably thiazolyl) any of which may be optionally substituted (preferably with one or more alkyl, or alkoxycarbonyl groups);

R³ is hydrogen or alkyl;

R⁴ is alkyl, optionally substituted heterocyclo, optionally substituted (aryl)alkyl (preferably substituted with a group of the formula —SO₂-alkyl), or optionally substituted (heteroaryl)alkyl;

or R³and R⁴ together with the nitrogen atom to which they are attached may combine to form an optionally substituted heterocyclo ring; (preferably piperadinyl, piperazinyl or morpholinyl);

R⁵ is alkyl or optionally substituted (aryl)alkyl (preferably substituted with one or more alkoxy or group of the formula —SO₂-alkyl); and

R⁶ is hydrogen.

More preferred compounds within Formula Ib are those wherein:

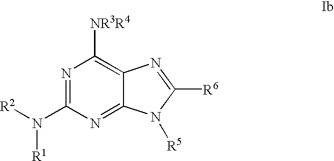

R¹ is hydrogen.

R² is

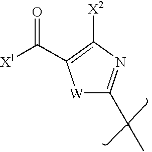

where W is O or S (preferably S), X¹ is alkoxy, and X² is alkyl, or 4-substituted aryl

R³ is hydrogen or alkyl;

R⁴ alkyl, optionally substituted heterocyclo, optionally substituted (aryl)alkyl (preferably substituted with a group of the formula —SO₂-alkyl), or optionally substituted (heteroaryl)alkyl;

or R³ and R⁴ together with the nitrogen atom to which they are attached may combine to form an optionally substituted heterocyclo ring; (preferably morpholinyl);

R⁵ is alkyl or optionally substituted (aryl)alkyl (preferably substituted with one or more alkoxy or group of the formula —SO₂-alkyl); and

R⁶ is hydrogen.

Further preferred compounds of formula Ib are chosen such that R⁴ or R⁵ or both R⁴ and R⁵ are optionally substituted (aryl)alkyl (preferably substituted with a group of the formula —SO₂-alkyl, —SO2-NH₂ or 3,4-dimethoxy), or optionally substituted (heteroaryl)alkyl (preferably optionally substituted (pyridyl)alkyl); Preferred compounds within formula I include:

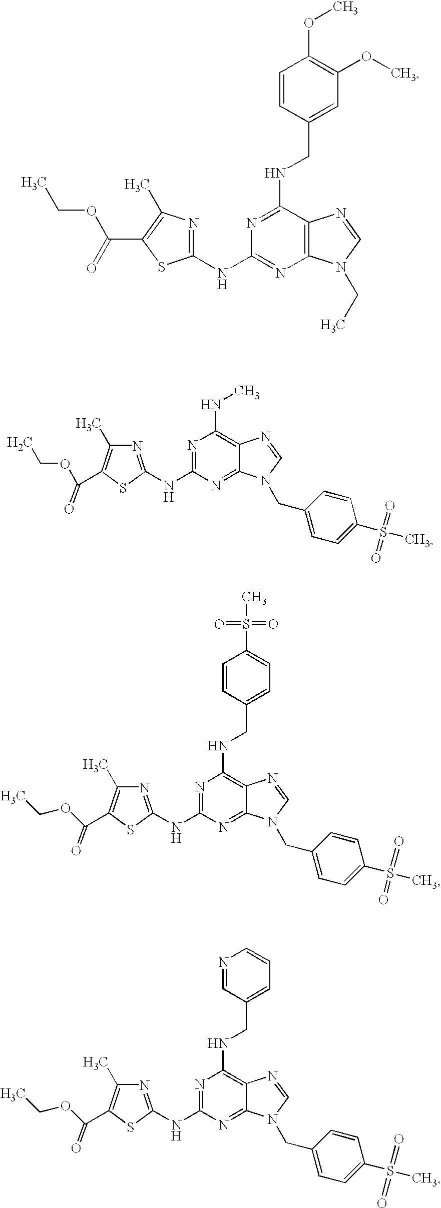

Additionally, compounds within the scope of the present invention include compounds of Formula II, pharmaceutically acceptable salts, prodrugs and solvates thereof:

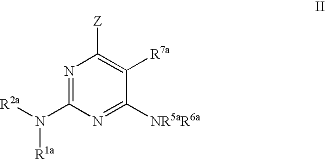

wherein

R^{1a} is H or alkyl;

R^{2a} is optionally substituted heteroaryl;

Z is halogen, alkyl, substituted alkyl, haloalkyl, or NR^{3a}R^{4a};

R^{3a} is hydrogen or alkyl;

R^{4a} is alkyl, optionally substituted (heteroaryl)alkyl, optionally substituted heterocylo, optionally substituted (heterocyclo)alkyl, or (aryl)alkyl wherein the aryl group is substituted with one or two groups T^{1*} and T^{2*} and optionally further substituted with a group T^{3*};

or R^{3a} and R^{4a} together with the nitrogen atom to which they are attached may combine to form an optionally substituted heterocyclo ring;

R^{5a} is (aryl)alkyl wherein the aryl group is substituted with one or two groups T^{1*} and T^{2*} and optionally further substituted with a group T^{3*};

R^{6a} is hydrogen or alkyl;

R^{7a} is hydrogen or alkyl;

T^{1*} and T^{2*} are independently alkoxy, alkoxycarbonyl, heteroaryl or —SO₂R^{8*} where R^{8a} is alkyl, amino, alkylamino or dialkylamino;

or T^{1*} and T^{2*} together with the atoms to which they are attached may combine to form a ring (e.g., benzodioxole);

T^{3*} is H, alkyl, halo, haloalkyl or cyano.

Preferred compounds within Formula II are those wherein:

R^{1a} is H;

R^{2a} is thiazolyl, oxazolyl, tetrahydroindolinyl, or isoxozolyl (preferably thiazolyl) any of which may be optionally substituted (preferably with one or more alkyl, alkylcarbonyl or alkoxycarbonyl groups);

Z is halogen, alkyl, haloalkyl, or NR^{3a}R^{4a};

R^{3a} is hydrogen;

R^{4a} is alkyl, haloalkyl, or optionally substituted (heterocyclo)alkyl, especially (morpholinyl)alkyl;

or R^{3a} and R^{4a} together with the nitrogen atom to which they are attached may combine to form an optionally substituted heterocyclo ring, especially piperazine optionally substituted with one or more alkyl or alkoxycarbonyl;

R^{5a} is

a) (phenyl)alkyl where the phenyl group is substituted with one or two alkoxy, alkoxycarbonyl, heteroaryl (especially thiadiazolyl) or —SO₂R^{8a}; or

b) optionally substituted (benzodioxole)alkyl, especially (1,3-benzodioxole)alkyl;

R^{6a} is hydrogen; and

R^{7a} is hydrogen or alkyl.

More preferred compounds within Formula II are those wherein:

R^{1a} is hydrogen.

R^{2a} is

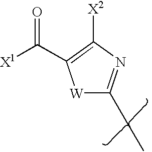

where W is O or S (preferably S), X¹ is alkoxy, and X² is alkyl;

Z is halogen, haloalkyl, or NR^{3a}R^{4a};

R^{3a} is hydrogen;

R^{4a} is alkyl, or optionally substituted (morpholinyl)alkyl;

or R^{3a} and R^{4a} together with the nitrogen atom to which they are attached may combine to form a piperazine ring optionally substituted with one or more alkyl or alkoxycarbonyl;

R^{5a} is

c) (phenyl)alkyl where the phenyl group is substituted with one or more alkoxy, alkoxycarbonyl, heteroaryl (especially thiadiazolyl) or —SO₂R^{8a}; or

d) optionally substituted (benzodioxole)alkyl, especially (1,3-benzodioxole)alkyl;

R^{6a} is hydrogen; and

R^{7a} is hydrogen or alkyl.

Preferred compounds within the scope of formula II include:

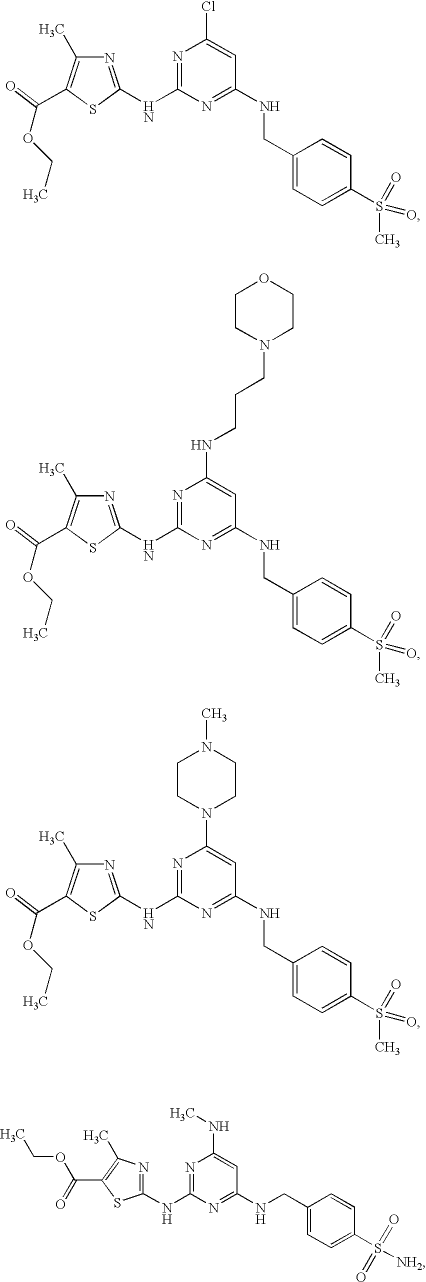

Additionally, compounds within the scope of the present invention include compounds of Formula III, pharmaceutically acceptable salts, prodrugs and solvates thereof:

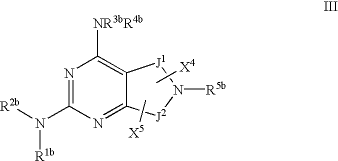

wherein

R^{1b} is H or alkyl;

R^{2b} is optionally substituted heteroaryl;

R^{3b} is H or alkyl;

R^{4b} is optionally substituted (aryl)alkyl;

R^{5b} is H, alkyl, or —C(O)—(CH₂)ᵥ—O—Y—R^{6b}, where Y is a bond or —C(O)—, R^{6b} is hydrogen or alkyl, and v is an integer from 0 to 2;

J¹ and J² are independently optionally substituted C₁₋₃ alkylene, provided that J¹ and J² are not both greater than C₂ alkylene;

X⁴ and X⁵ are optional substituents bonded to any available carbon atom in one or both of J¹ and J², independently selected from hydrogen, OR⁷, NR⁸R⁹, alkyl, substituted alkyl, alkenyl, substituted alkenyl, alkynyl, substituted alkynyl, cycloalkyl, substituted cycloalkyl, aryl, substituted aryl, heterocycloalkyl, or heteroaryl;

R⁷ is hydrogen, alkyl, substituted alkyl, alkenyl, alkynyl, cycloalkyl, substituted cycloalkyl, C(O)alkyl, C(O)substituted alkyl, C(O)cycloalkyl, C(O) substituted cycloalkyl, C(O)aryl, C(O)substituted aryl, C(O)Oalkyl, C(O)Osubstituted alkyl, C(O)heterocycloalkyl, C(O)heteroaryl, aryl, substituted aryl, heterocycloalkyl and heteroaryl; and

R⁸ and R⁹ are independently selected from the group consisting of hydrogen, alkyl, substituted alkyl, cycloalkyl, substituted cycloalkyl, alkenyl, alkynyl, C(O)alkyl, C(O)substituted alkyl, C(O)cycloalkyl, C(O)substituted cycloalkyl, C(O)aryl, C(O)substituted aryl, C(O)Oalkyl, C(O)Osubstituted alkyl, C(O)heterocycloalkyl, C(O)heteroaryl, S(O)₂alkyl, S(O)₂substituted alkyl, S(O)₂cycloalkyl, S(O)₂substituted cycloalkyl, S(O)₂aryl, S(O)₂substituted aryl, S(O)₂heterocycloalkyl, S(O)₂heteroaryl, aryl, substituted aryl, heterocycloalkyl, and heteroaryl, or R₈ and R₉ taken together with the nitrogen atom to which they are attached complete an optionally substituted heterocycloalkyl or heteroaryl ring.

Preferred compounds within the scope of formula III include compounds of formula IIa and IIIb

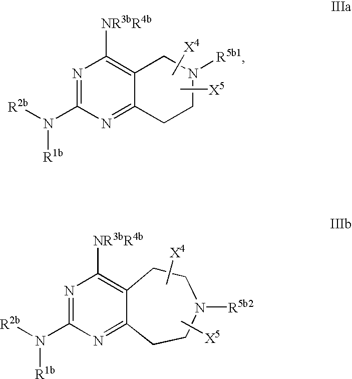

wherein

R^{1b}, R^{2b}, R^{3b}, R^{4b}, X⁴ and X⁵ are as defined above;

R^{5b1} is H or alkyl; and

R^{5b2} is —C(O)—(CH₂)ᵥ—O—Y—R^{6b}, where Y is a bond or —C(O)—, R^{6b} is hydrogen or alkyl, and v is an integer from 0 to 2;

Preferred compounds within Formula III are those wherein:

R^{1b} is H;

R^{2b} is thiazolyl, oxazolyl, or isoxozolyl (preferably thiazolyl) any of which may be optionally substituted (preferably with one or more alkyl, or alkoxycarbonyl groups);

R^{3b} is H;

R^{4b} is optionally substituted (pheny)alkyl, (preferably substituted with one or more group of the formula —SO₂R^{8b} where R^{8b} is alkyl, amino, alkylamino or dialkylamino);

R^{5b} is alkyl, or —C(O)—(CH₂)ᵥ—O—Y—R^{6b}, where Y is a bond or —C(O)—, R^{6b} is hydrogen or alkyl, and v is 1;

J¹ is an alkylene group of 1 or 2 carbon atoms;

J² is an alkylene group of 2 carbon atoms; and

X⁴ and X⁵ are each H.

More preferred compounds within Formula III are those wherein

R^{1b} is H;

R^{2b} is

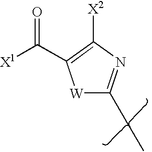

where W is O or S (preferably S), X¹ is alkoxy, and X² is alkyl;

R^{3b} is H;

R^{4b} is (pheny)alkyl substituted with one or more group of the formula —SO₂R^{8b} where R^{8b} is alkyl, or amino;

R^{5b} is alkyl, or —C(O)—(CH₂)ᵥ—O—Y—R^{6b}, where Y is a bond or —C(O)—, R^{6b} is hydrogen or alkyl, and v is 1;

J¹ is an alkylene group of 1 or 2 carbon atoms;

J² is an alkylene group of 2 carbon atoms; and

X⁴ and X⁵ are each H.

Preferred compounds within the scope of Formula III include:

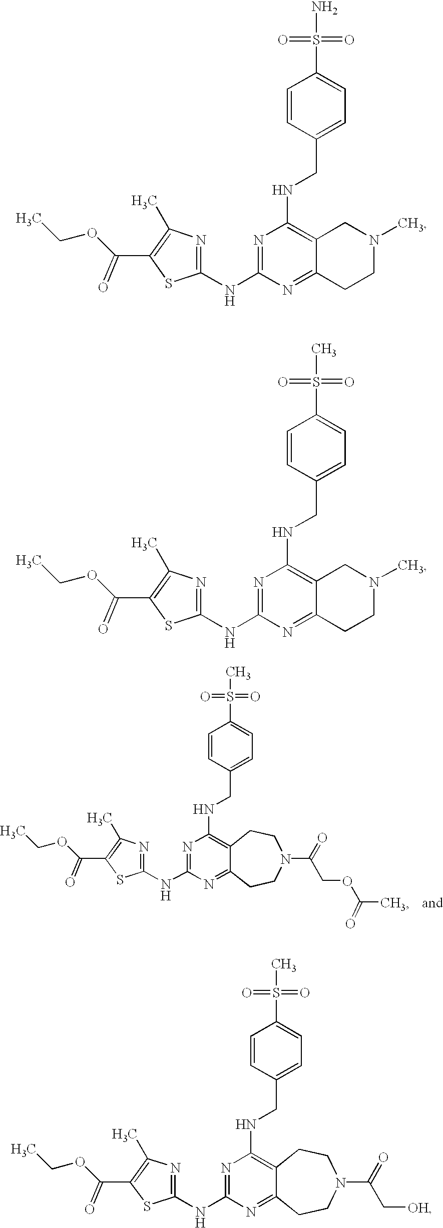

Additionally, compounds within the scope of the present invention include to compounds of Formula IV, pharmaceutically acceptable salts, prodrugs and solvates thereof:

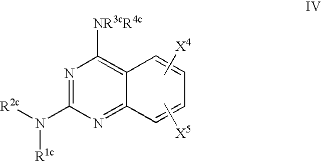

wherein

R^{1c} is H or alkyl;

R^{2c} is optionally substituted heteroaryl;

R^{3c} is H or alkyl;

R^{4c} is optionally substituted (aryl)alkyl; and

X⁴ and X⁵ are as defined in Formula III.

Preferred compounds within Formula IV are those wherein:

R^{cb} is H;

R^{2c} is thiazolyl, oxazolyl, or isoxozolyl (preferably thiazolyl) any of which may be optionally substituted (preferably with one or more alkyl, or alkoxycarbonyl groups);

R^{3c} is H;

R^{4c} is optionally substituted (pheny)alkyl, (preferably substituted with one or more group of the formula —SO₂R^{8c} where R^{8c} is alkyl, amino, alkylamino or dialkylamino); and

X⁴ and X⁵ are each H.

More preferred compounds within Formula IV are those wherein

R^{1c} is H;

R^{2c} is

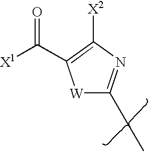

where W is O or S (preferably S), X¹ is alkoxy, and X² is alkyl;

R^{3c} is H;

R^{4c} is (pheny)alkyl substituted with one or more group of the formula —SO₂R^{8c} where R^{8c} is amino; and

X⁴ and X are each H.

Preferred compounds within the scope of Formula IV include:

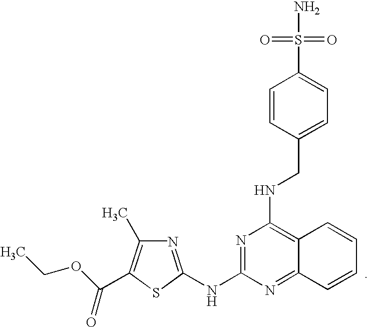

The following are definitions of the terms as used throughout this specification and claims.

A dual PDE7-PDE4 inhibitor (PDE4/7 or PDE7/4) is defined herein as any compound which has an IC₅₀ in both a PDE7 and a PDE4 inhibition assay of less than 20 micromolar (preferably less than 10 micromolar, and most preferably less than 5 micromolar), and an IC₅₀ in a PDE3 inhibition assay which is at least 10 times higher than the IC₅₀ of the compound in the PDE7 assay (more preferably at least 20 times higher than the IC₅₀ of the compound in the PDE7 assay, and most preferably at least 100 times higher than the IC₅₀ of the compound in the PDE7 assay). Preferred dual PDE7-PDE4 inhibitors include those that inhibit PDE3, PDE4 and PDE7 as described above, and further inhibit PDE1 at an IC₅₀ at least 10 times higher than the IC₅₀ of the compound in a PDE7 assay (more preferably at least 20 times higher than the IC₅₀ of the compound in the PDE7 assay, and most preferably at least 100 times higher than the IC₅₀ of the compound in the PDE7 assay). Preferred dual PDE7-PDE4 inhibitors further include those compounds that inhibit PDE3, PDE4 and PDE7 as described above, and further suppress both T cell proliferation, and TNF-alpha secretion from either THP-1 monocytes or human peripheral blood mononuclear cell at a level of less than 20 micromolar.

A selective PDE7 inhibitor is defined herein as a compound for which the IC₅₀ of the compound in a PDE7 inhibition assay is less than 20 micro molar (preferably less than 10 micromolar, more preferably less than 5 micromolar, most preferably less than 1 micromolar). The PDE7 IC₅₀ of a selective PDE7 inhibitor should be less than one-tenth the IC₅₀ of said compound in all of the following PDE assays: PDE1, PDE3 and PDE4 (more preferably the PDE7 IC₅₀ of a selective PDE7 inhibitor should be less than one-twentieth the IC₅₀ of said compound in the following PDE assays: PDE1 and PDE3, most preferably the PDE7 IC₅₀ of a selective PDE7 inhibitor should be less than one-hundreth the IC₅₀ of said compound in a PDE3 assay).

A selective PDE4 inhibitor is defined herein as a compound for which the IC₅₀ of the compound in a PDE4 inhibition assay is less than 20 micromolar (preferably less than 10 micromolar, more preferably less than 5 micromolar, most preferably less than 1 micromolar), and doesn't inhibit PDE7 with an IC₅₀ of less than 10 times the IC₅₀ of said compound in a PDE4 assay or doesn't inhibit PDE7 with an IC₅₀ of less than 1 micromolar. Examples of selective PDE4 inhibitors currently in development include Arofyline, Cilomilast, Roflumilast, C-11294A, CDC-801, BAY-19-8004, Cipamfylline, SCH351591, YM-976, PD-189659, Mesiopram, Pumafentrine, CDC-998, IC-485, and KW-4490.

“Leukocyte activation” is defined herein as any or all of leukocyte (T cell, monocyte macrophage, neutrophil etc.) cell proliferation, cytokine production, adhesion protein expression, and production of inflammatory mediators. This is mediated in part by the action of PDE4 and/or PDE7 depending on the particular leukocyte under consideration.

The terms “alk” or “alkyl” refer to straight or branched chain hydrocarbon groups having 1 to 12 carbon atoms, preferably 1 to 8 carbon atoms, such as methyl, ethyl, n-propyl, i-propyl, n-butyl, i-butyl, t-butyl, pentyl, hexyl, heptyl, octyl, etc. Lower alkyl groups, that is, alkyl groups of 1 to 6 carbon atoms, are generally most preferred.

The term “substituted alkyl” refers to alkyl groups substituted with one or more groups listed in the definition of T¹, T² and T³, preferably selected from halo, cyano, O—R₇, S—R₇, NR₈R₉, nitro, cycloalkyl, substituted cycloalkyl, oxo, aryl, substituted aryl, heterocyclo, heteroaryl, CO₂R₇, S(O)R₇, SO₂R₇, SO₃R₇, SO₂NR₈R₉, C(O)NR₈R₉, C(O)alkyl, and C(O)H.

The term “alkylene” refers to a straight chain bridge of 1 to 4 carbon atoms connected by single bonds (e.g., —(CH₂)_{X}— wherein x is 1 to 5), which may be substituted with one or more groups listed in the definition of T¹, T² and T³.

The term “alkenyl” refers to straight or branched chain hydrocarbon groups having 2 to 12 carbon atoms, preferably 2 to 4 carbon atoms, and at least one double carbon to carbon bond (either cis or trans), such as ethenyl.

The term “substituted alkenyl” refers to an alkenyl group as defined above substituted with one or more groups listed in the definition of T¹, T² and T³, preferably selected from halo, cyano, O—R₇, S—R₇, NR₈R₉, nitro, cycloalkyl, substituted cycloalkyl, oxo, aryl, substituted aryl, heterocyclo, heteroaryl, CO₂R₇, S(O)R₇, SO₂R₇, SO₃R₇, SO₂NR⁸R₉, C(O)NR₈₉, C(O)alkyl, and C(O)H.

The term “alkynyl” refers to straight or branched chain hydrocarbon group having 2 to 12 carbon atoms and one, two or three triple bonds, preferably 2 to 6 carbon atoms and one triple bond.

The term “substituted alkynyl” refers to an alkynyl group as defined above substituted with one or more groups listed in the definition of T¹, T² and T³, preferably selected from halo, cyano, O—R₇, S—R₇, NR₈R₉, nitro, cycloalkyl, substituted cycloalkyl, oxo, aryl, substituted aryl, heterocyclo, heteroaryl, CO₂R₇, S(O)R₇, SO₂R₇, SO₃R₇, SO₂NR₈R₉, C(O)NR₈R₉, C(O)alkyl, and C(O)H.

The term “halo” refers to chloro, bromo, fluoro, and iodo.

The term “cycloalkyl” refers to saturated and partially unsaturated (containing 1 or 2 double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 7 carbons, forming the ring and which may be fused to 1 or 2 aromatic or heterocyclo rings, which include cyclopropyl, cyclobutyl, cyclopentyl, cyclohexyl, cycloheptyl, cyclooctyl, cyclodecyl, cyclododecyl, cyclohexenyl,

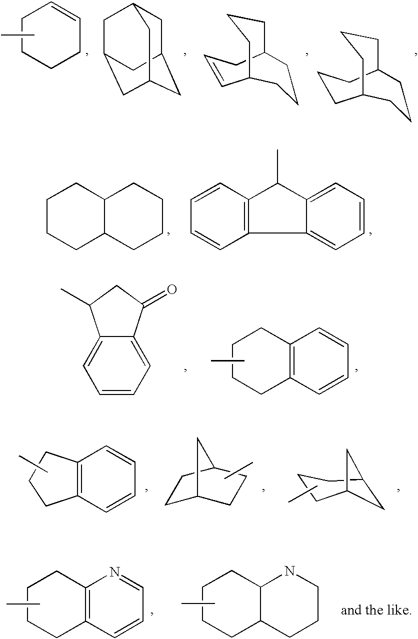

The term “substituted cycloalkyl” refers to such cycloalkyl group as defined above substituted with one or more groups listed in the definition of T¹, T² and T³, preferably selected from halogen, nitro, alkyl, substituted alkyl, alkenyl, cyano, cycloalkyl, substituted cycloalkyl, aryl, substituted aryl, heterocyclo, heteroaryl, oxo, OR₇, CO₂R₇, C(O)NR₈R₉, OC(O)R₇, OC(O)OR₇, OC(O)NR₈R₉, OCH₂CO₂R₇, C(O)R₇, NR₈R₉, NR₁₀C(O)R₇, NR₁₀C(O)OR₇, NR₁₀C(O)C(O)OR₇, NR₁₀C(O)C(O)NR₈R₉, NR₁₀C(O)C(O)alkyl, NR₁₀C(NCN)OR₇, NR₁₀C(O)NR₈R₉, NR₁₀C(NCN)NR₈R₉, NR₁₀C(NR₁₁)NR₈R₉, NR₁₀SO₂NR₈R₉, NR₁₀SO₂R₇, SR₇, S(O)R₇, SO₂R₇, SO₃R₇, SO₂NR₈R₉, NHOR₇, NR₁₀NR₈R₉, N(COR₇)₁₀, N(CO₂R₇)OR₁₀, C(O)NR₁₀(CR₁₂R₁₃)ᵣR₇, CO(CR₁₂R₁₃)pO(CR₁₄R₁₅)qCO₂R₇, CO(CR₁₂R₁₃)rOR₇, CO(CR₁₂R₁₃)pO(CR₁₄R₁₅)qR₇, CO(CR₁₂R₁₃)rNR₈R₉, OC(O)O(CR₁₂R₁₃)mNR₈R₉, OC(O)N(CR¹²R₁₃)rR₇, O(CR₁₂R₁₃)mNR₈R₉, NR₁₀C(O)(CR₁₂R₁₃)rR₇, NR₁₀C(O)(CR₁₂R₁₃)rOR₇, NR₁₀C(═NC)(CR₁₂R₁₃)rR₇, NR₁₀CO(CR₁₂R₁₃)rNR₈R₉, NR₁₀(CR₁₂R₁₃)mOR₇, NR₁₀(CR₁₂R₁₃)rCO₂R₇, NR₁₀(CR₁₂R₁₃)mNR₈R₉, NR₁₀(CR₁₂R₁₃)nSO₂(CR¹⁴R₁₅)qR₇, CONR₁₀(CR₁₂R₁₃)nSO₂(CR₁₄R₁₅)qR₇, SO₂NR₁₀(CR₁₂R₁₃)nCO(CR₁₄R₁₅)qR₇, and SO₂NR₁₀(CR₁₂R₁₃)mOR₇,

The terms “ar” or “aryl” refer to aromatic homocyclic (i.e., hydrocarbon) mono-, bi- or tricyclic ring-containing groups preferably having 6 to 12 members such as phenyl, naphthyl and biphenyl, as well as such rings fused to a cycloalkyl, cycloalkenyl, heterocyclo, or heteroaryl ring. Examples include:

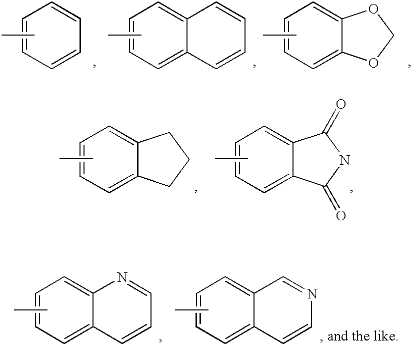

The term “substituted aryl” refers to such aryl groups as defined above substituted with one or more groups listed in the definition of T¹, T² and T³, preferably selected from halogen, nitro, alkyl, substituted alkyl, alkenyl, cyano, cycloalkyl, substituted cycloalkyl, aryl, substituted aryl, heterocyclo, heteroaryl, OR₇, CO₂R₇, C(O)NR₈R₉, OC(O)R₇, OC(O)OR₇, OC(O)NR₈R₉, OCH₂CO₂R₇, C(O)R₇, NR₈R₉, NR₁₀C(O)R₇, NR₁₀C(O)OR₇, NR₁₀C(O)C(O)OR₇, NR₁₀C(O)C(O)NR₈R₉, NR₁₀C(O)C(O)alkyl, NR₁₀C(NCN)OR₇, NR₁₀C(O)NR₈R₉, NR₁₀C(NCN)NR₈R₉, NR₁₀C(NR₁₁)NR₈R₉, NR₁₀SO₂NR₈R₉, NR₁₀SO₂R₇, SR₇, S(O)R₇, SO₂R₇, SO₃R₇, SO₂NR₈R₉, NHOR₇, NR₁₀NR₈R₉, N(COR₇)OR₁₀, N(CO₂R₇)OR₁₀, C(O)NR₁₀(CR₁₂R₁₃)ᵣR₇, CO(CR₁₂R₁₃)pO(CR₁₄R₁₅)qCO₂R₇, CO(CR₁₂R₁₃)rOR₇, CO(CR₁₂R₁₃)pO(CR₁₄R₁₅)qR₇, CO(CR₁₂R₁₃)rNR₈R₉, OC(O)O(CR₁₂R₁₃)mNR₈R₉, OC(O)N(CR₁₂R₁₃)rR₇, O(CR₁₂R₁₃)mNR₈R₉, NR₁₀C(O)(CR₁₂R₁₃)rR₇, NR₁₀C(O)(CR₁₂R₁₃)rOR₇, NR₁₀C(═NC)(CR₁₂R₁₃)rR₇, NR₁₀CO(CR₁₂R₁₃)rNR₈R₉, NR₁₀(CR₁₂R₁₃)mOR₇, NR₁₀(CR₁₂R₁₃)rCO₂R₇, NR₁₀(CR₁₂R₁₃)mNR₈R₉, NR₁₀(CR₁₂R₁₃)nSO₂(CR₁₄R₁₅)qR₇, CONR₁₀(CR₁₂R₁₃)nSO₂(CR₁₄R₁₅)qR₇, SO₂NR₁₀(CR₁₂R₁₃)nCO(CR₁₄R₁₅)qR₇, and SO₂NR₁₀(CR₁₂R₁₃)mOR₇ as well as pentafluorophenyl.

The terms “heterocycle”, “heterocyclic”, “heterocyclic group” or “heterocyclo” refer to fully saturated or partially unsaturated cyclic groups (for example, 3 to 13 member monocyclic, 7 to 17 member bicyclic, or 10 to 20 member tricyclic ring systems, preferably containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. The rings of multi-ring heterocycles may be either fused, bridged and/or joined through one or more spiro unions. Exemplary heterocyclic groups include

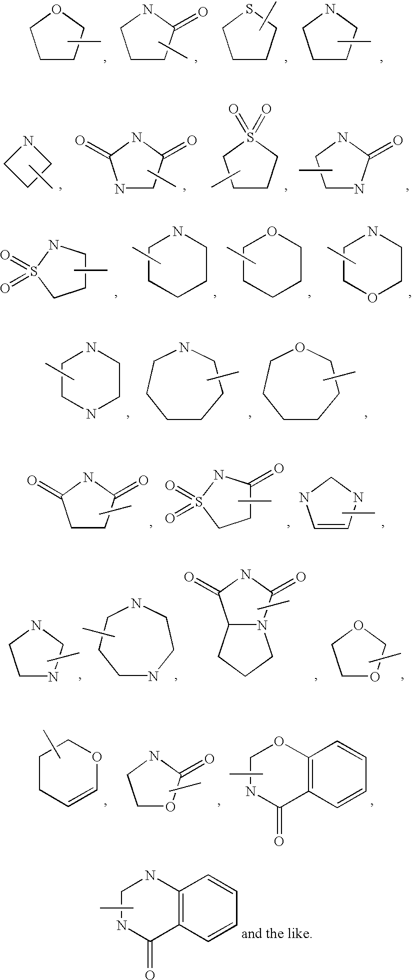

The terms “substituted heterocycle” or “substituted heterocyclo” and the like refer to such heterocylo groups as defined above substituted with one or more groups listed in the definition of T¹, T² and T³, preferably selected from halogen, nitro, alkyl, substituted alkyl, alkenyl, cyano, cycloalkyl, substituted cycloalkyl, aryl, substituted aryl, heterocyclo, heteroaryl, oxo, OR₇, CO₂R₇, C(O)NR₈R₉, OC(O)R₇, OC(O)OR₇, OC(O)NR₈R₉, OCH₂CO₂R₇, C(O)R₇, NR₈R₉, NR₁₀C(O)R₇, NR₁₀C(O)OR₇, NR₁₀C(O)C(O)OR₇, NR₁₀C(O)C(O)NR₈R₉, NR₁₀C(O)C(O)alkyl, NR₁₀C(NCN)OR₇, NR₁₀C(O)NR₈R₉, NR₁₀C(NCN)NR₈R₉, NR₁₀C(NR₁₁)NR₈R₉, NR₁₀SO₂NR₈R₉, NR₁₀SO₂R₇, SR₇, S(O)R₇, SO₂R₇, SO₃R₇, SO₂NR₈R₉, NHOR₇, NR₁₀NR₈R₉, N(COR₇)OR₁₀, N(CO₂R₇)OR₁₀, C(O)NR₁₀(CR₁₂R₁₃)ᵣR₇, CO(CR₁₂R₁₃)pO(CR₁₄R₁₅)qCO₂R₇, CO(CR₁₂R₁₃)rOR₇, CO(CR₁₂R₁₃)pO(CR₁₄R₁₅)qR₇, CO(CR₁₂R₁₃)rNR₈R₉, OC(O)O(CR₁₂R₁₃)mNR₈R₉, OC(O)N(CR₁₂R₁₃)rR₇, O(CR₁₂R₁₃)mNR₈R₉, NR₁₀C(O)(CR₁₂R₁₃)rR₇, NR₁₀C(O)(CR₁₂R₁₃)rOR₇, NR₁₀C(═NC)(CR₁₂R₁₃)rR₇, NR₁₀CO(CR₁₂R₁₃)rNR₈R₉, NR₁₀(CR₁₂R₁₃)mOR₇, NR₁₀(CR₁₂R₁₃)rCO₂R₇, NR₁₀(CR₁₂R₁₃)mNR₈R₉, NR₁₀(CR₁₂R₁₃)nSO₂(CR₁₄R₁₅)qR₇, CONR₁₀(CR₁₂R₁₃)nSO₂(CR₁₄R₁₅)qR₇, SO₂NR₁₀(CR₁₂R₁₃)nCO(CR₁₄R₁₅)qR₇, and SO₂NR₁₀(CR₁₂R₁₃)mOR₇.

The term “heteroaryl” as used herein alone or as part of another group refers to a 5- 6- or 7-membered aromatic rings containing from 1 to 4 nitrogen atoms and/or 1 or 2 oxygen or sulfur atoms provided that the ring contains at least 1 carbon atom and no more than 4 heteroatoms. The heteroaryl ring is linked through an available carbon or nitrogen atom. Also included within the definition of heteroaryl are such rings fused to a cycloalkyl, aryl, cycloheteroalkyl, or another heteroaryl ring. One, two, or three available carbon or nitrogen atoms in the heteroaryl ring can be optionally substituted with substituents listed in the description of T₁, T₂ and T₃. Also an available nitrogen or sulfur atom in the heteroaryl ring can be oxidized. Examples of heteroaryl rings include

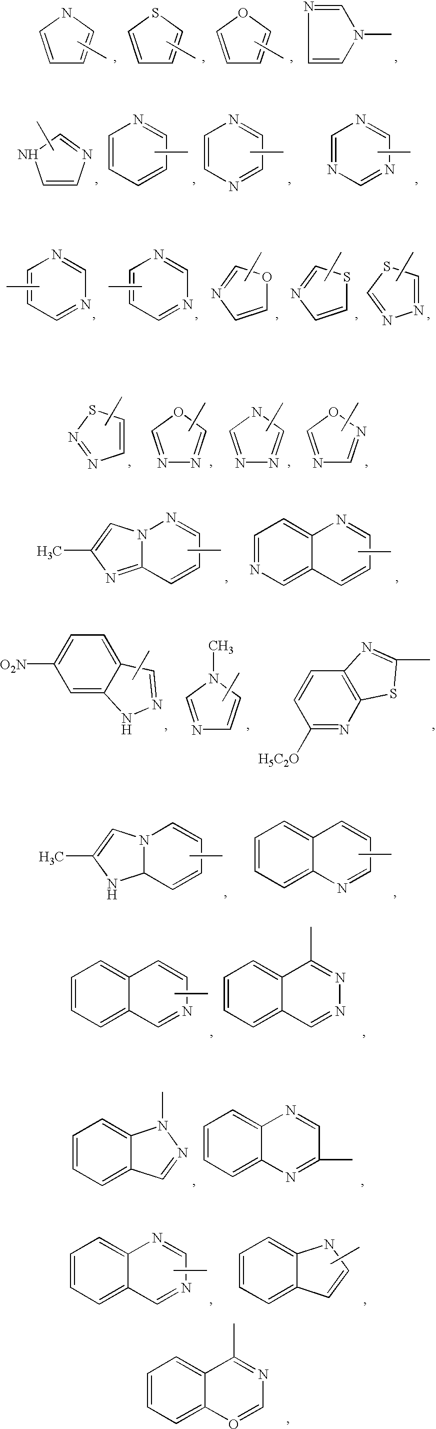

The term “substituted heteroaryl” refers to such heteroaryl groups as defined above substituted on any available atom with one or more groups listed in the definition of T¹, T² and T³, preferably selected from” refers to such heterocylo groups as defined above substituted with one or more groups listed in the definition of T¹, T² and T³, preferably selected from halogen, nitro, alkyl, substituted alkyl, alkenyl, cyano, cycloalkyl, substituted cycloalkyl, aryl, substituted aryl, heterocyclo, heteroaryl, OR₇, CO₂R₇, C(O)NR₈R₉, OC(O)R₇, OC(O)OR₇, OC(O)NR₈R₉, OCH₂CO₂R₇, C(O)R₇, NR₈R₉, NR₁₀C(O)R₇, NR₁₀C(O)OR₇, NR₁₀C(O)C(O)OR₇, NR₁₀C(O)C(O)NR₈R₉, NR₁₀C(O)C(O)alkyl, NR₁₀C(NCN)OR₇, NR₁₀C(O)NR₈R₉, NR₁₀C(NCN)NR₈R₉, NR₁₀C(NR₁₁)NR₈R₉, NR₁₀SO₂NR₈R₉, NR₁₀SO₂R₇, SR₇, S(O)R₇, SO₂R₇, SO₃R₇, SO₂NR₈R₉, NHOR₇, NR₁₀NR₈R₉, N(COR₇)OR₁₀, N(CO₂R₇)OR₁₀, C(O)NR₁₀(CR₁₂R₁₃)ᵣR₇, CO(CR₁₂R₁₃)pO(CR₁₄R₁₅)qCO₂R₇, CO(CR₁₂R₁₃)rOR₇, CO(CR₁₂R₁₃)pO(CR₁₄R₁₅)qR₇, CO(CR₁₂R₁₃)rNR₈R₉, OC(O)O(CR₁₂R₁₃)mNR₈R₉, OC(O)N(CR₁₂R₁₃)rR₇, O(CR₁₂R₁₃)mNR₈R₉, NR₁₀C(O)(CR₁₂R₁₃)rR₇, NR₁₀C(O)(CR₁₂R₁₃)rOR₇, NR₁₀C(═NC)(CR₁₂R₁₃)rR₇, NR₁₀CO(CR₁₂R₁₃)rNR₈R₉, NR₁₀(CR₁₂R₁₃)mOR₇, NR₁₀(CR₁₂R₁₃)rCO₂R₇, NR₁₀(CR₁₂R₁₃)mNR₈R₉, NR₁₀(CR₁₂R₁₃)nSO₂(CR₁₄R₁₅)qR₇, CONR₁₀(CR₁₂R₁₃)nSO₂(CR₁₄R₁₅)qR₇, SO₂NR₁₀(CR₁₂R₁₃)nCO(CR₁₄R₁₅)qR₇, and SO₂NR₁₀(CR₁₂R₁₃)mOR₇.

R₇, R₁₀, and R₁₁, are independently selected from the group consisting of hydrogen, alkyl, substituted alkyl, alkenyl, alkynyl, cycloalkyl, substituted cycloalkyl, C(O)alkyl, C(O)substituted alkyl, C(O)cycloalkyl, C(O) substituted cycloalkyl, C(O)aryl, C(O)substituted aryl, C(O)Oalkyl, C(O)Osubstituted alkyl, C(O)heterocyclo, C(O)heteroaryl, aryl, substituted aryl, heterocyclo and heteroaryl.

R₈ and R₉ are independently selected from the group consisting of hydrogen, alkyl, substituted alkyl, cycloalkyl, substituted cycloalkyl, alkenyl, alkynyl, C(O)alkyl, C(O)substituted alkyl, C(O)cycloalkyl, C(O)substituted cycloalkyl, C(O)aryl, C(O)substituted aryl, C(O)Oalkyl, C(O)Osubstituted alkyl, C(O)heterocyclo, C(O)heteroaryl, S(O)₂alkyl, S(O)₂substituted alkyl, S(O)₂cycloalkyl, S(O)₂substituted cycloalkyl, S(O)₂aryl, S(O)₂substituted aryl, S(O)₂heterocyclo, S(O)₂heteroaryl, aryl, substituted aryl, heterocyclo, and heteroaryl or R₈ and R₉ taken together with the nitrogen atom to which they are attached complete a heterocyclo or heteroaryl ring.

R₁₂ and R₁₄ are independently selected from hydrogen and alkyl or 1 to 4 carbons.

R₁₃ and R₁₅ are independently selected from hydrogen, alkyl of 1 to 4 carbons, and substituted alkyl or 1 to 4 carbons.

n is zero or an integer from 1 to 4.

m is an integer from 2 to 6.

p is an integer from 1 to 3.

q is zero or an integer from 1 to 3.

r is zero or an integer from 1 to 6.

T¹, T², and T³ are are each independently

(1) hydrogen or T⁶, where T⁶ is

(i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;

(ii) (ii) a group (i) which is itself substituted by one or more of the same or different groups (i); or

(iii) (iii) a group (i) or (ii) which is independently substituted by one or more (preferably 1 to 3) of the following groups (2) to (13) of the definition of T¹, T² and T³,

(2) —OH or —OT⁶,

(3) —SH or —ST⁶,

(4) —C(O)ₜH, —C(O)ₜT⁶, or —O—C(O)T⁶, where t is 1 or 2;

(5) —SO₃H, —S(O)ₜT⁶, or S(O)ₜN(T⁹)T⁶,

(6) halo,

(7) cyano,

(8) nitro,

(9) -T⁴-NT⁷T⁸,

(10) -T⁴-N(T⁹)-T⁵-NT⁷T⁸,

(11) -T⁴-N(T¹⁰)-T⁵-T⁶,

(12) -T⁴-N(T¹⁰)-T⁵-H,

(13) oxo,

T⁴ and T⁵ are each independently

(1) a single bond,

(2) -T¹¹-S(O)ₜT¹²-,

(3) -T¹¹-C(O)-T¹²-,

(4) -T¹¹-C(S)-T¹²-,

(5) -T¹¹-O-T¹²-,

(6) -T¹¹-S-T¹²-,

(7) -T¹¹-O—C(O)-T¹²-,

(8) -T¹¹-C(O)—O-T¹²-,

(9) -T¹¹-C(═NT^{9a})-T¹²-, or

(10) -T¹¹-C(O)—C(O)-T¹²-T⁷, T⁸, T⁹, T^{9a} and T¹⁰

(1) are each independently hydrogen or a group provided in the definition of T⁶, or

(2) T⁷ and T⁸ may together be alkylene or alkenylene, completing a 3- to 8-membered saturated or unsaturated ring together with the atoms to which they are attached, which ring is unsubstituted or substituted with one or more groups listed in the description of T¹, T² and T³, or

(3) T⁷ or T⁸, together with T⁹, may be alkylene or alkenylene completing a 3- to 8-membered saturated or unsaturated ring together with the nitrogen atoms to which they are attached, which ring is unsubstituted or substituted with one or more groups listed in the description of T¹, T² and T³, or

(4) T⁷ and T⁸ or T⁹ and T¹⁰ together with the nitrogen atom to which they are attached may combine to form a group —N═CT¹³T¹⁴ where T¹³ and T¹⁴ are each independently H or a group provided in the definition of T⁶; and T¹¹ and T¹² are each independently

(1) a single bond,

(2) alkylene,

(3) alkenylene, or

(4) alkynylene.

Dual PDE7-PDE4 inhibitors (including the compounds of formula I, II, III or IV) in accordance with the present invention are employed, typically in the form of a pharmaceutical composition including a pharmaceutically acceptable carrier for the treatment of leukocyte activation-associated, or leukocyte activation-mediated disorders. The compounds employed for this purpose are typically administered in an amount from about 0.01 to 100 mg/kg/day.

The pharmaceutical compositions comprising at least one dual PDE7-PDE4 inhibitor may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The dual PDE7-PDE4 inhibitors may be administered by any suitable means, for example, orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered in the form of liposomes.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The present compounds may also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

The effective amount of a compound employed in the present invention may be determined by one of ordinary skill in the art, and includes exemplary dosage amounts for an adult human of from about 0.01 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to leukocyte activation-associated, or leukocyte activation-mediated disorders.

Compounds of Formulas I, II, III and IV include salts, prodrugs and solvates. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the R substituents comprise an acid moiety such as a carboxyl group). Also included herein are quaternary ammonium salts such as alkylammonium salts. Pharmaceutically acceptable (i.e., non-toxic, physiologically acceptable) salts are preferred, although other salts are useful, for example, in isolation or purification steps which may be employed during preparation. Salts of the compounds of the formula I may be formed, for example, by reacting a compound I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts (formed, for example, where the R substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. The basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug”, as employed herein, denotes a compound which, upon administration to a subject, undergoes chemical conversion by metabolic or chemical processes to yield a compound of the Formulas I, II, III or IV or a salt and/or solvate thereof. Solvates of the compounds of Formulas I, II, III or IV are preferably hydrates.

All stereoisomers of the present compounds, such as those which may exist due to asymmetric carbons on the R substituents of the compound of the formulas I, II, III or IV including enantiomeric and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

### Methods of Preparation

Compounds of Formulas I, II, III or IV may be prepared by reference to the methods illustrated in the following Schemes A through C. As shown therein the end product is a compound having the same structural formula as Formulas I, II, III or IV. It will be understood that any compound of Formulas I, II, III or IV may be produced by Scheme A and B by the suitable selection of appropriate substitution. Schemes C shows the preparation of amides from compounds of Formulas I, II, III or IV derived from Schemes A and B. Solvents, temperatures, pressures, and other reaction conditions may readily be selected by one of ordinary skill in the art. All documents cited are incorporated herein by reference in their entirety. Starting materials are commercially available or readily prepared by one of ordinary skill in the art. Constituents of compounds are as defined herein or elsewhere in the specification.

The methods described herein may be carried out with starting materials and/or reagents in solution or alternatively, where appropriate, with one or more starting materials or reagents bound to a solid support (see (1) Thompson, L. A., Ellman, J. A., Chemical Reviews, 96, 555-600 (1996); (2) Terrett, N. K., Gardner, M., Gordon, D. W., Kobylecki, R. J., Steele, J., Tetrahedron, 51, 8135-8173 (1995); (3) Gallop, M. A., Barrett, R. W., Dower, W. J., Fodor, S. P. A., Gordon, E. M., Journal of Medicinal Chemistry, 37, 1233-1251 (1994); (4) Gordon, E. M., Barrett, R. W., Dower, W. J., Fodor, S. P. A., Gallop, M. A., Journal of Medicinal Chemistry, 37, 1385-1401 (1994); (5) Balkenhohl, F., von dem Bussche-Hüinnefeld, Lansky, A., Zechel, C., Angewandte Chemie International Edition in English, 35, 2288-2337 (1996); (6) Balkenhohl, F., von dem Bussche-Hüinnefeld, Lansky, A., Zechel, C., Angewandte Chemie, 108, 2436-2487 (1996); and (7) Sofia, M. J., Drugs Discovery Today, 1, 27-34 (1996)).

Scheme A illustrates a general method for the solid phase preparation of compounds of Formula I. Solid supports enable a molecule of interest to be synthesized with facile removal of reagents and is used by one skilled in the art as an alternative to the conventional synthesis of compounds in solution. A starting Compound I anchored to a suitable resin (such as a SASRIN resin, as indicated by the darkened sphere) can be treated with a reducing agent such as sodium cyanoborohydride in the presence of an amine II to give an amine III. Coupling with a appropriate dichloroheterocyle, in this case a dichloropurine, derivative IV in the presence of a base such as diisopropyl ethyl amine in a solvent such as N-methyl pyrrolidone gives substituted purine V. Conversion of V under palladium-catalyzed coupling conditions in the presence of an amine VI gives the resin anchored Compound VII. Cleavage from the resin using acidic conditions such as TFA gives compound VIII which are examples of compounds of formula Ia.

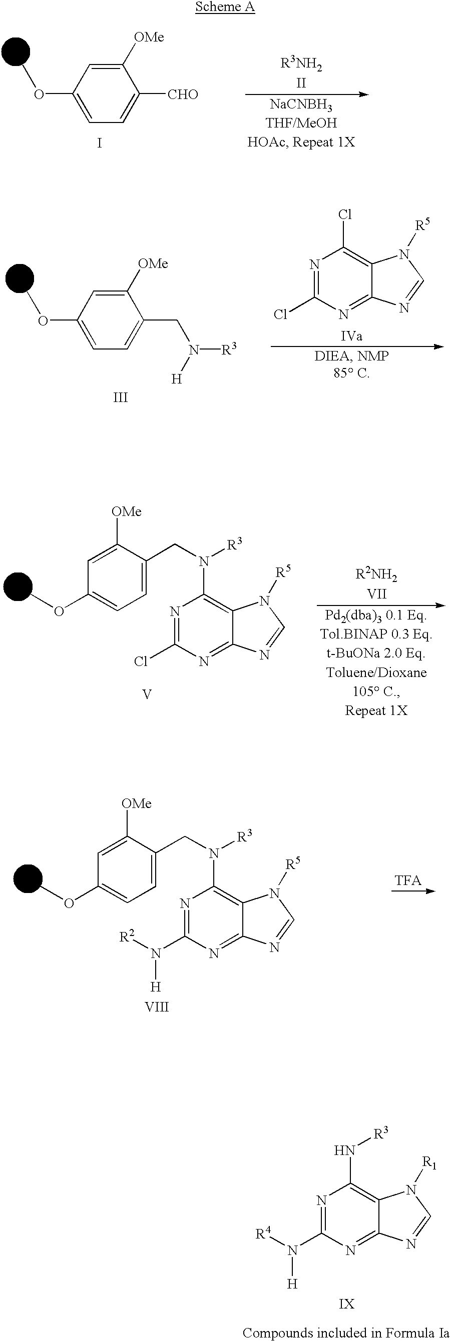

Scheme B 1 outlines the solution phase synthesis of compounds of Formulas Ia and Ib. Compound X is treated with an alkyl halide in the presence of a base such as potassium carbonate in acetone to give a mixture of Compounds IVa and IVb. Separation of the isomers is accomplished using standard chromatography techniques. The intermediate IVa or IVb may be reacted with reagent XI, which may be an or an alcohol, a thiol or a sulfonamide on the presence of a suitable base to provide intermediate XII. Conversion of XII under palladium-catalysed coupling conditions in the presence of an amine XIII gives compound IX.

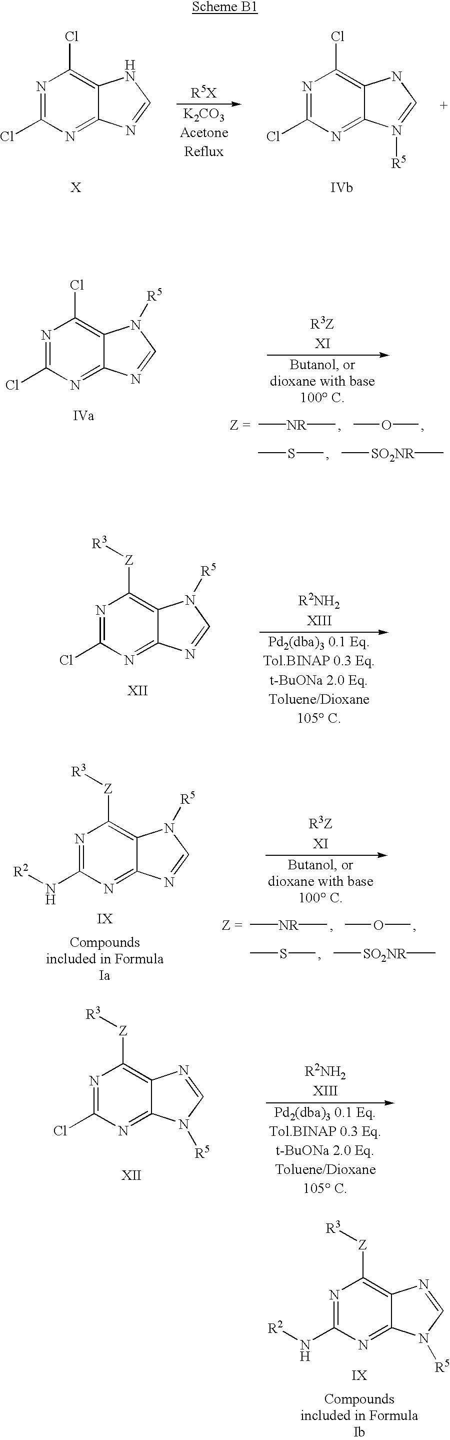

The procedure illustrated in Scheme B1 can be used with compound IVb to produce compounds of formula IIb. The method outlined in scheme B1 is general for a number of chloroheterocyles with minor changes which would be readily apparent to one skilled in the art of organic chemistry.

Scheme B2 illustrates the synthesis of quinazolines of formulas IV. Dichlorointermediate XIV is reacted with reagent XI, which may be an or an alcohol, a thiol or a sulfonamide on the presence of a suitable base to provide intermediate XV

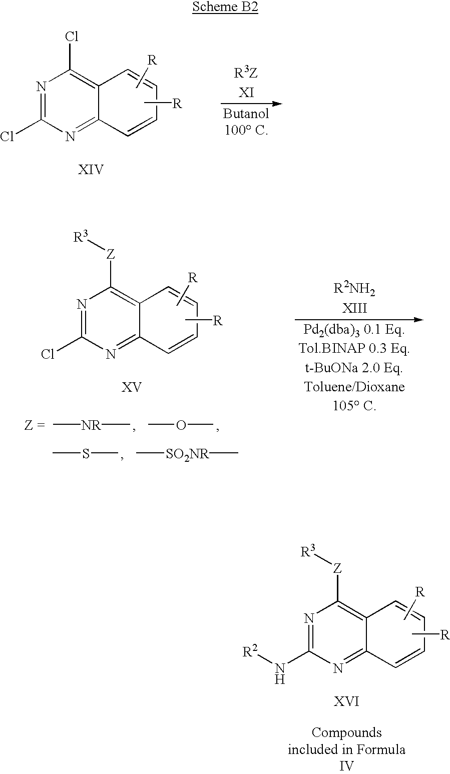

Compounds of formula II may be prepared from readily available starting materials by a number of methods known to one skilled in the art of organic chemistry and is illustrated in Scheme B3. An amine is reacted with reagent XVII to provide guanidine XVII which is deprotected and free based to yield guanidine XIX. Reaction with either beta-keto ester XX, or a malonate XX with heat with or without added base condenses to produce pyrimidine XXI. This pyrimidine is reacted with phosphorous oxychloride to produce intermediate pyrimidine XXII. Reaction with reagent XI, which may be an or an alcohol, a thiol or a sulfonamide on the presence of a suitable base to provide pyrimidines XXIII, which are compounds of Formula III. In the case of pyrimidine XXIIIa, the chloro group may be replaced by an amine by reaction at elevated temperature, or, in some cases with the aid of a microwave apparatus, to produce pyrimidine XXIV which are also compounds of Formula III.

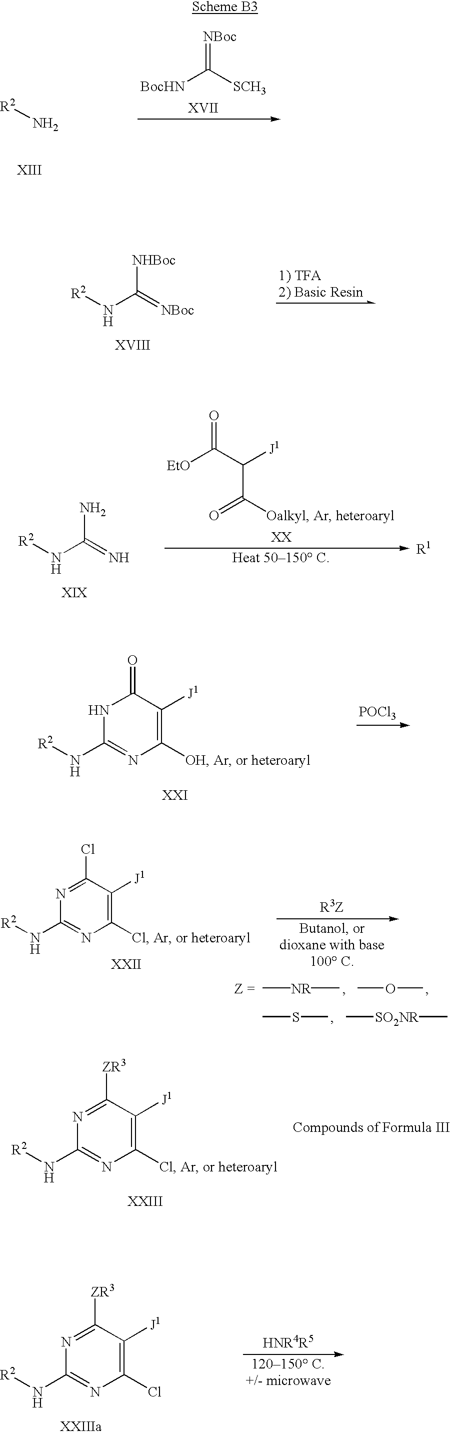

In some instances the intermediate guanidines XIX might be readily prepared by direct synthesis, an example of which is illustrated in scheme B3.1. alpha-Haloketone XXV is reacted with a thiobiuret such as XXVI to provide the guanidine salt XXVII, which is liberated by treatment with a basic resin, or sodium hydroxide, sodium methoxide, or an amine base to provide intermediate XIXa, which can be further elaborated to compounds of formula III as illustrated in scheme B1

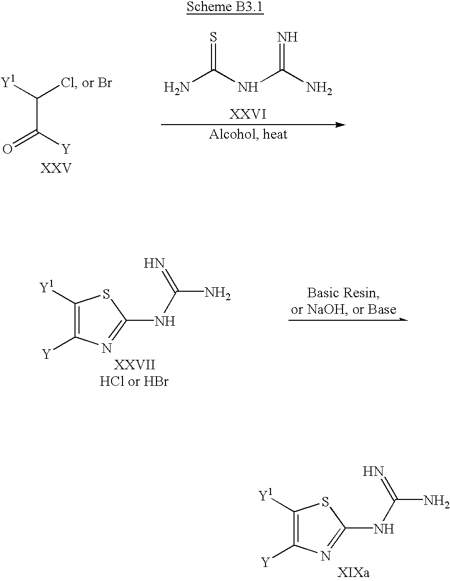

### Scheme B4

A number of heterocycles may be prepared by applying cyclic beta-keto esters to the synthesis illustrated in Scheme B3. In this case guanidine XIX is heated with a cyclic beta-keto ester XXVIII to produce intermediate XXIX. Reaction with phosphorous oxychloride provides intermidiate XXX. Reaction with reagent XI, which may be an amine, an alchol, a thiol or a sulfonamide on the presence of a suitable base to provide compound XXXI which is a compound of formula III.

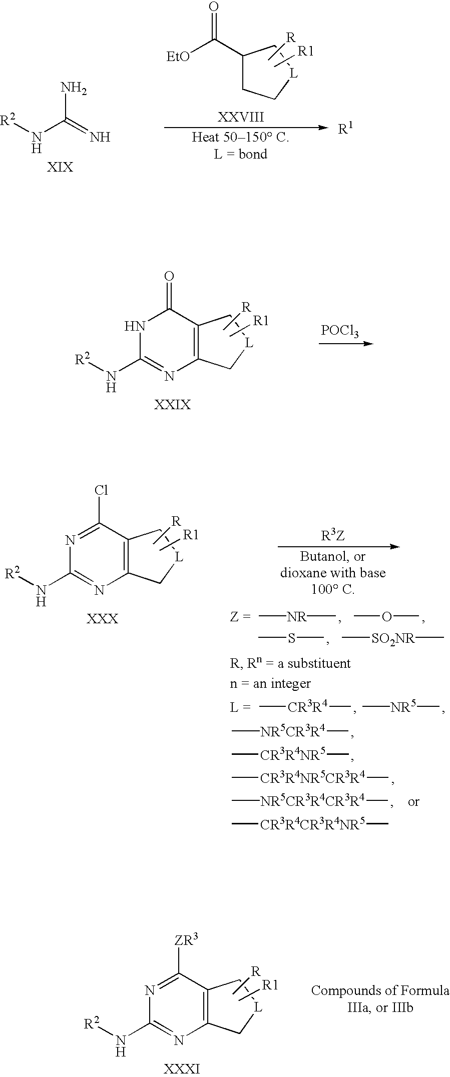

Cyclic beta-keto esters of structure XXVIII, are either commercially available, or readily prepared by one of the methods outlined in Schemes B4.1 and B4.2 In scheme B4.1 an amine XXXII is reacted with dialkylacrylate XXXIII to provide the di-addition product XXXIV. Reaction with a base such as sodium alkoxide results in a Dieckmann cyclization to produce XXVIIIa

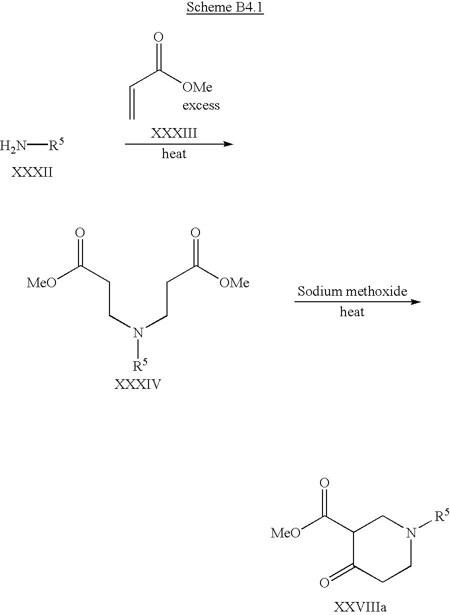

Seven member cyclic beta-keto esters of structure XXVIIIb, can be prepared from piperidones XXXV, which are either commercially available or can be prepared by a number of methods, including decarboxylation of XXVIIIIa with reagents such as sodium bromide at elevated temperature. Treatment of the piperidone with ethyl diazoacetate and boron trifluoride etherate at reduced temperature provide the ring expanded intermediate XXVIIIb, useful for the preparation of compounds of formula VIIIa.

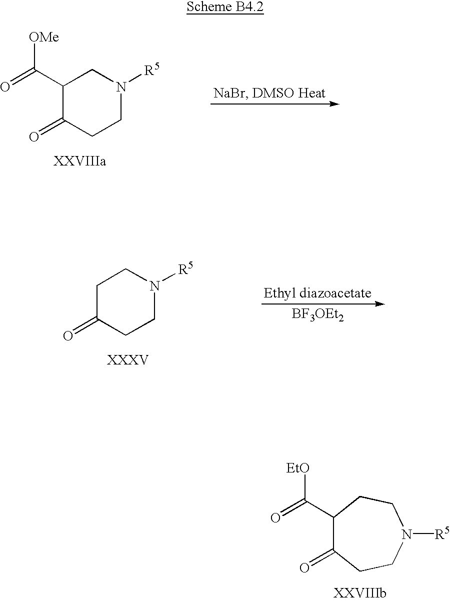

Scheme C outlines the conversion of esters of Formula I to amides of Formula I. Hydrolysis of the ester of Compound IX under basic conditions such as sodium hydroxide affords the acid XXXVI. Coupling of XXXVI under standard amide bond coupling techniques (DIC/HOAt) with the appropriate amine XXXVII gives the desired amide XXXVIII.

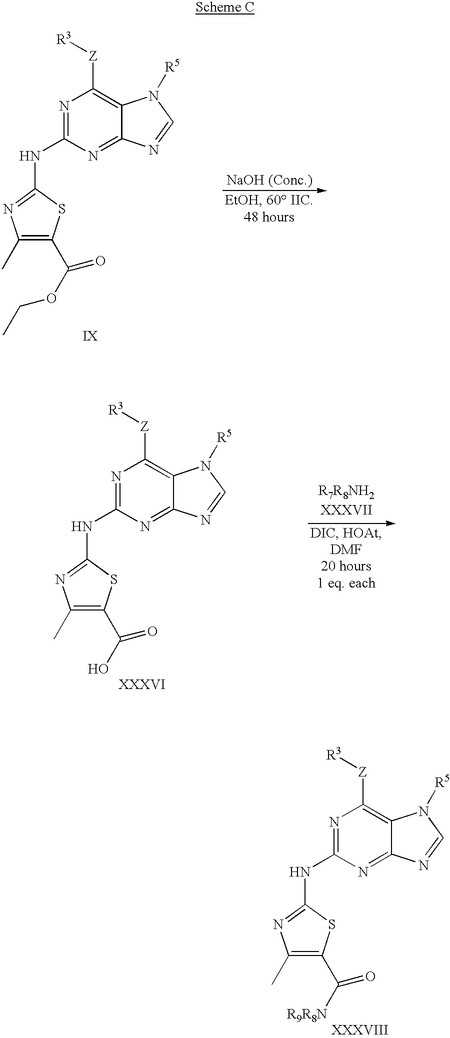

### Utility

Dual PDE7-PDE4 inhibitors (including compounds of formulas I, II, III and IV) are useful in the treatment (including prevention, partial alleviation or cure) of leukocyte activation-associated disorders, which include (but are not limited to) disorders such as: transplant rejection (such as organ transplant, acute transplant, xenotransplant or heterograft or homograft such as is employed in bum treatment); protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; respiratory and pulmonary diseases including but not limited to asthma, exercise induced asthma, chronic obstructive pulmonary disease (COPD), emphysema, bronchitis, and acute respiratory distress syndrome (ARDS); inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (e.g., asthma, hayfever, allergic rhinitis) or skin allergies; scleracierma; mycosis fungoides; acute inflammatory and respiratory responses (such as acute respiratory distress syndrome and ishchemia/reperfusion injury); dermatomyositis; alopecia areata; chronic actinic dermatitis; eczema; Behcet's disease; Pustulosis palmoplanteris; Pyoderma gangrenum; Sezary's syndrome; atopic dermatitis; systemic schlerosis; and morphea.

The term “leukocyte activation-associated disorder” as used herein includes each of the above referenced diseases or disorders. The compounds of the present invention are useful for treating the aforementioned exemplary disorders irrespective of their etiology.

Those present compounds which are dual PDE7/4 inhibitors may be more effective than either a selective PDE4 inhibitor or a selective PDE7 inhibitor in the above mentioned disease states, as a result of either additive or synergistic activity resulting from the combined inhibition of PDE7 and PDE4. Additionally, the simultaneous or sequential co-administration of a selective PDE4 inhibitor together with a selective PDE7 inhibitor would be expected to approximate the activity of a dual PDE7/4 inhibitor.

The present invention thus provides methods for the treatment of disorders as discussed above comprising the step of administering to a subject in need thereof of at least one dual PDE7-PDE4 inhibitor for the treatment of leukocyte activation-associated or leukocyte-activation mediated disease. Other therapeutic agents such as those described below may be employed with the compounds of the present invention. In the methods of the present invention, such other therapeutic agent(s) may be administered prior to, simultaneously with or following the administration of the compound(s) of the present invention.

Exemplary of such other therapeutic agents which may be used in combination with dual PDE7-PDE4 inhibitors include the following: cyclosporins (e.g., cyclosporin A), CTLA4-Ig, antibodies such as anti-ICAM-3, anti-IL-2 receptor (Anti-Tac), anti-CD45RB, anti-CD2, anti-CD3, anti-CD4, anti-CD80, anti-CD86, monoclonal antibody OKT3, agents blocking the interaction between CD40 and CD154, such as antibodies specific for CD40 and/or CD154 (i.e., CD40L), fusion proteins constructed from CD40 and CD154 (CD40Ig and CD8-CD154), inhibitors, such as nuclear translocation inhibitors, of NF-kappa B function, such as deoxyspergualin (DSG), non-steroidal antiinflammatory drugs (NSAIDs) such as ibuprofen, steroids such as prednisone or dexamethasone, gold compounds, antiproliferative agents such as methotrexate, FK506 (tacrolimus, Prograf), mycophenolate mofetil, cytotoxic drugs such as azathiprine and cyclophosphamide, TNF-α inhibitors such as tenidap, anti-TNF antibodies or soluble TNF receptor such as etanercept (Enbrel), rapamycin (sirolimus or Rapamune), leflunomide (Arava), beta-2 agonists such as albuterol, levalbuterol (Xopenex), and salmeterol (Serevent), inhibitors of leukotriene synthesis such as montelukast (Singulair) and zariflukast (Accolate), and anticholinergic agents such as ipratropium (Atrovent) and cyclooxygenase-2 (COX-2) inhibitors such as celecoxib (Celebrex) and rofecoxib (Vioxx), or derivatives thereof, anti-cytokines such as anti-IL-1 mAb or IL-1 receptor agonist, anti-IL-4 or IL-4 receptor fusion proteins and PTK inhibitors such as those disclosed in the following U.S. Patent Applications, incorporated herein by reference in their entirety: Ser. No. 60/056,770, filed Aug. 25, 1997 (Attorney Docket No. QA202*), Ser. No. 60/069,159, filed Dec. 9, 1997, Ser. No. 09/097,338, filed Jun. 15, 1998 (Attorney Docket No. QA202b), Ser. No. 60/056,797, filed Aug. 25, 1997, Ser. No. 09/094,797, filed Jun. 15, 1998 (Attorney Docket No. QA205a), Ser. No. 60/065,042, filed Nov. 10, 1997 (Attorney Docket No. QA207*), Ser. No. 09/173,413, filed Oct. 15, 1998, Ser. No. 60,076,789, filed Mar. 4, 1998, and Ser. No. 09,262,525, filed Mar. 4, 1999.

Alternatively a selective PDE7 inhibitor may be co-administered with a selective PDE4 inhibitor such as Arofyline, Cilomilast, Rofltumilast, C-11294A, CDC-801, BAY-19-8004, Cipamfylline, SCH351591, YM-976, PD-189659, Mesiopram, Pumafentrine, CDC-998, IC-485, and KW-4490. Other selective PDE4 inhibitors are well known in the literature, and include compounds disclosed in the following patent documents: US 20020013467, WO 0200609, WO 0164648, WO 0164647, WO 0157036, WO 0157036, WO 0147915, WO 0147914, WO 0147905, WO 0147880, WO 0147879, WO 0146184, WO, 0146172, WO 0142244, WO 0111967, U.S. Pat. No. 5,591776, WO 9808844, and WO 9808830. Selective PDE7 inhibitors have been disclosed in the literature, such as IC242, (Lee, et. al. PDE7A is expressed in human B-lymphocytes and is up-regulated by elevation of intracellular cAMP. Cell Signalling, 14, 277-284, (2002)) and also include compounds disclosed in the following patent documents: WO 0068230, WO 0129049, WO 0132618, WO 0134601, WO 0136425, WO 0174786, WO 0198274, U.S. Provisional Application Serial No. 60/287,964, and U.S. Provisional Application Serial No. 60/355,141. Selective PDE7 inhibitors further include the compounds of Examples F1 and F2 herein.

The above other therapeutic agents, when employed in combination with the compounds of the present invention, may be used, for example, in those amounts indicated in the Physicians' Desk Reference (PDR) or as otherwise determined by one of ordinary skill in the art.

### PDE-containing Cell Lysates

Hut78 cells were grown in 10% FCS in Iscoves Modified Dulbecco's Medium (Gibco BRL-Life Technologies, Grand Island, N.Y.) with antibiotics. Cells were centrifuged and resuspended in four volumes of [40 mM Tris (pH 7.5)/50 μM EDTA/200 μM PMSF with a cocktail of Protease inhibitors (Boehringher Mannheim, Indianapolis, Ind.)] at 4 C. Cells were homogenized using a Virtis homogenizer, and the lysate was centrifuged twice for 15 min at 15,000×g. Glycerol was added to a final volume of 50% for storage at −20C.

### SPA Assay

Inhibition of PDE activity in Hut78 cell lysate was determined using an SPA specific for cAMP (Amersham Pharmacia Biotech, Buckinghamshire, UK) according to the manufacturers instructions with minor modifications. Enzyme assays were performed at room temperature in the presence of 50 mM Tris HCl, pH7.5, containing 8.3 mM MgCl₂, 1.7 mM EGTA and 0.5 mg/mL BSA. Each assay was performed in a 100 μL reaction volume in 96 well microtitre plates containing the above buffer, 0.3 ul of Hut78 cell lysate treated with 2 uM Zardaverine to inhibit PDE3 and PDE4, 0.05 uCi of [5′,8-₃H] Adenosine 3′,5′-cyclic phosphate as an ammonium salt for 20 min. The reaction was terminated by the addition of 50 μl PDE SPA beads (1 mg) water with 10 mM cold cAMP (Sigma, St. Louis Mo.). The reaction mix was allowed to settle for 20 minutes before counting in a Top Count-NXT scintillation counter (Packard BioScience, Meriden, Conn.). For individual PDE enzymes other than PDE7, the assay was essentially unchanged except that ³H-cyclic GMP was used as the substrate for PDE1, PDE5 and PDE6. The following PDEs/activators and enzyme sources were used: PDE1, bovine (Sigma St Louis), calmodulin; PDE2, rat kidney, cGMP; PDE3, human platelet; PDE4, rat kidney; PDE5, human platelet, and PDE6, bovine retina.

### T Cell Proliferation Assay

Peripheral blood mononuclear cells (PBMC) were isolated from whole blood by density gradient centrifugation over Lymphoprep, 1.077. Cells were plated into 96 well U-bottom plates at 2.5×10₅ cells/well in 10% FBS RPMI 1640 (Life Technologies/Gibco-BRL) containing 10 ug/ml anti-CD3 (G19-4, Bristol-Myers Squibb P.R.I., Princeton, N.J.) and lug/ml anti-CD28 (9.3, Bristol-Myers Squibb P.R.I.) in the presence and absence of inhibitors. DMSO (used as a solvent for inhibitors) was added to the medium at 0.1% final concentration. The total volume per well was 200 μL. Cells were incubated at 37 C. 5% CO2 for 3 days, at which time 0.5 μCi of ³H-thymidine was added to each well. Six hours following the addition of ³H-thmidine, the plates were harvested onto filter plates, 30 ul EcoLite scintillant (ICN, Costa Mesa, Calif.) was added per well, and plates read on a Top Count-NXT scintillation counter.

### TNFA Secretion Assay

The ability of compounds to inhibit the production and secretion of TNFα from leukocytes was performed using either PBMC (obtained as described above) or the THP-1 cell line as a source of monocytes. Compounds were diluted in RPMI 1640 supplemented with 10% FBS and DMSO at a final concentration of 0.2%. Cells (2×10⁵/well in U-bottom 96 well plates) were pre-incubated with compounds for 30 min at 37 C. prior to addition of lipopolysaccharide (LPS) at a final concentration of 6.25 ng/ml in a total volume of 200 μL. After 4 h at 37 C., 50 μL of supernatant was carefully aspirated for detection of soluble TNFα. Soluble TNFα was detected by ELISA developed by R&D Systems (Minneapolis, Minn.) according to the manufacturers instructions.

### EXAMPLES

The following examples illustrate preferred embodiments of the present invention and do not limit the scope of the present invention. Abbreviations employed in the Examples are defined below. Compounds of the Examples are identified by the example and step in which they are prepared (e.g., “A1.1” denotes the title compound of step 1 of Example A1), or by the example only where the compound is the title compound of the example (for example, “A2” denotes the title compound of Example A2).

Abbreviations

| | |
|---|---|
| Ac | Acetyl |
| AcOH | Acetic acid |
| aq. | Aqueous |
| CDI | Carbonyldiimidazole |
| Bn | Benzyl |
| Bu | Butyl |
| Boc | tert-butoxycarbonyl |
| DMAP | Dimethylaminopyridine |
| DMA | N,N-Dimethylacetamide |
| DMF | dimethylformamide |
| DMSO | Dimethylsulfoxide |
| EDC | 1-(3-Dimethylaminopropyl)-3-ethylcarbodiimide |
| | hydrochloride |
| EtOAc | Ethyl acetate |
| Et | Ethyl |
| EtOH | Ethanol |
| H | Hydrogen |
| h | Hours |
| i | iso |
| HPLC | High pressure liquid chromatography |
| HOAc | Acetic acid |
| Lawesson's Reagent | [2,4-bis(4-methoxyphenyl)-1,3-dithia-2,4- |
| | diphosphetane-2-4-disufide |
| LC | liquid chromatography |
| Me | Methyl |
| MeOH | Methanol |
| min. | Minutes |
| M⁺ | (M + H)⁺ |
| M⁺¹ | (M + H)⁺ |
| MS | Mass spectrometry |
| n | normal |
| Pd/C | Palladium on carbon |
| Ph | Phenyl |
| Pr | Propyl |
| Ret Time | Retention time |
| rt or RT | Room temperature |
| sat. | Saturated |
| S-Tol-BINAP | (S)-(-)-2,2′-Bis(di-p-tolylphosphino)-1,1′-binapthyl |
| t | tert |
| TFA | Trifluoroacetic acid |
| THF | Tetrahydrofuran |
| YMC | YMC Inc. Wilmington, NC 28403 |

HPLC conditions used to determine retention times; 2 min gradient 0-100% B in A(A; 0.1% TFA in 90/10 water/methanol; B; 0.1% TFA in 10/90 water/methanol) using a YMC turbopack column at with a detection wavelength of 220 nanometers or 254 nanometers.

### Example A1

### 4-Methyl-2-[[6-(methylamino-9-[[4-(methylsulfonyl)phenyl]methyl]-9H-purin-2-yl]amino]-5-thiazolecarboxylic Acid Ethyl Ester

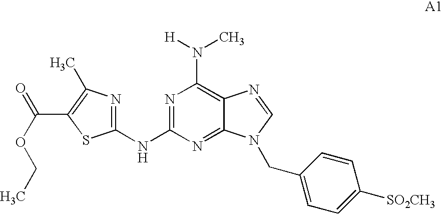

A1.1: 2,6-Dichloro-9-(4-methylsulfonylbenzyl)purine

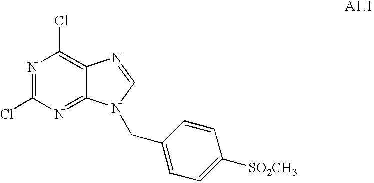

Potassium carbonate (823 mg, 5.95 mmol, 4.5 eq) was added to a solution of 2,6-dichloropurine (250 mg, 1.32 mmol, 1 eq) in N,N-dimethylformamide (13 mL) and the resultant mixture was stirred at rt for 20 min before 4-methylsulfonylbenzyl chloride (541 mg, 2.64 mmol, 2 eq) was added. After stirring for 46 h at rt, the reaction mixture was filtered and the filtrate was concentrated in vacuo and purified by column chromatography [acetone/ethyl acetate/hexanes=1:1:2 (v/v)] to afford 304 mg (64%) of A1.1, as a white solid. LC/MS: 358 [M+H]⁺; HPLC: >99% at 2.94 min (Phenomenex 5 μm C18 column 4.6×50 mm, 10-90% aqueous methanol over 4 min containing 0.2% phosphoric acid, 4 mL/min, monitoring at 254 nm); ¹H NMR (400 MHz, DMSO-d₆): δ8.87 (s, 1 H), 7.91 (d, J=8.3 Hz, 2 H), 7.57 (d, J=8.3 Hz, 2 H), 5.64 (s, 2 H), 3.20 (s, 3 H).

A1.2: 2-Chloro-6-(N-methylamino)-9-(4-methylsulfonylbenzyl)purine

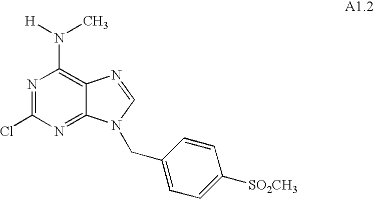

A mixture of 2,6-dichloro-9-(4-methylsulfonylbenzyl)purine (30 mg, 0.084 mmol, 1 eq), methylamine (8.03 M in ethanol, 21 μl, 0.168 mmol, 2 eq), and diisopropylethylamine (50 μL, 0.277 mmol, 3.3 eq) in 1-butanol (0.85 mL) was heated at 100° C. for 3 h. The reaction mixture was cooled to rt and the solid was collected by filtration, washed with cold methanol and dried to provide 22 mg (75%) of A1.2 as a slightly yellow solid. LC/MS: 352 [M+H]⁺; HPLC: >90% at 2.72 min (Phenomenex 5 μm C18 column 4.6×50 mm, 10-90% aqueous methanol over 4 min containing 0.2% phosphoric acid, 4 mL/min, monitoring at 254 nm); ¹H NMR (400 MHz, DMSO-d₆): δ8.31 (br s, 1 H), 8.29 (s, 1 H), 7.91 (d, J=8.3 Hz, 2 H), 7.48 (d, J=8.2 Hz, 2 H), 5.48 (s, 2 H), 3.19 (s, 3 H), 2.92 (d, J=4.3 Hz, 3 H).

A1.3: 4-Methyl-2-[[6-(methylamino)-9-[[4-(methylsulfonyl)phenyl]methyl]-9H-purin-2-yl]amino]-5-thiazolecarboxylic Acid Ethyl Ester

To a solution of A1.2 (35.6 mg, 0.101 mmol, 1 eq) and ethyl 2-amino-4-methylthiazole-5-carboxylate (37.7 mg, 0.202 mmol, 2 eq) in dimethylacetamide (1 mL) in a 1-dram vial was added tris(dibenzylideneacetone)dipalladium(0) (9.2 mg, 0.010 mmol, 0.1 eq), 2-(di-t-butylphosphino)biphenyl (9.0 mg, 0.030 mmol, 0.3 eq) and sodium t-butoxide (19.4 mg, 0.202 mmol, 2 eq). The vial was purged with N₂, sealed and heated in a 105° C. oil bath for 5 h. The reaction mixture was cooled to rt, filtered through celite and concentrated in vacuo. The residue was treated with methanol (ca. 1 mL) and the precipitated solid was collected by filtration, washed with methanol and dried to afford 30 mg (60%) of product as a tan solid. LC/MS: 502 [M+H]⁺; HPLC: >90% at 3.52 min (Phenomenex 5 μm C18 column 4.6×50 mm, 10-90% aqueous methanol over 4 min containing 0.2% phosphoric acid, 4 mL/min, monitoring at 254 nm); ¹H NMR (400 MHz, DMSO-d₆): δ11.55 (s, 1 H), 8.16 (s, 1 H), 8.00 (br s, 1 H), 7.89 (d, J=8.3 Hz, 2 H), 7.56 (d, J=8.0 Hz, 2 H), 5.47 (s, 2 H), 4.22 (q, J=7.0 Hz, 2 H), 3.16 (s, 3 H), 3.05 (br s, 3 H), 1.28 (t, J=7.0 Hz, 3 H).

### Example A2-A7

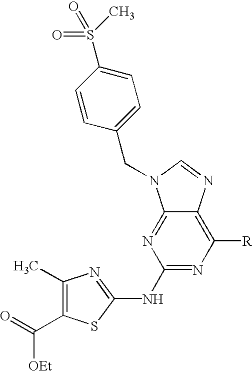

Examples A2 to A22 were prepared in a similar manner to that used for Example A1 with the exception that the appropriate amine was used in step C1.2.

TABLE A

| | | | HPLC | |
|---|---|---|---|---|
| | | | Retention^{a} | MS |
| Ex. | R | Name | (min) | Reported |
| A2 | 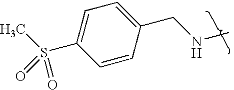 | 4-Methyl-2-[[9-[[4- (methylsulfonyl)phenyl]methyl]- 6-[[[4- (methylsulfonyl)phenyl]methyl]amino]-9H-purin-2-yl]amino]-5- thiazolecarboxylic acid ethyl ester | 3.20 | 656.12 |
| A3 | 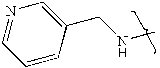 | 4-Methyl-2-[[9-[[4- (methylsulfonyl)phenyl]methyl]- 6-[(3-pyridinylmethyl)amino]- 9H-purin-2-yl]amino]-5- thiazolecarboxylic acid ethyl ester | 2.60 | 579.44 |
| A4 | 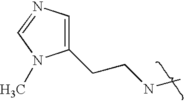 | 4-Methyl-2-[[6-[[2-(1-methyl- 1H-imidazol-5-yl)ethyl]amino]- 9-[[4- (methylsulfonyl)phenyl]methyl]- 9H-purin-2-yl]amino]-5- thiazolecarboxylic acid ethyl ester | 2.62 | 596.42 |
| A5 | 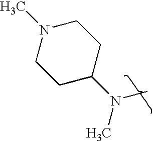 | 4-Methyl-2-[[6-[methyl(1- methyl-4-piperidinyl)amino]-9- [[4- (methylsulfonyl)phenyl]methyl]- 9H-purin-2-yl]amino]-5- thiazolecarboxylic acid ethyl ester | 2.75 | 599.19 |
| A6 | 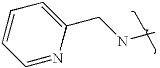 | 4-Methyl-2-[[9-[[4- (methylsulfonyl)phenyl]methyl]- 6-[(2-pyridinylmethyl)amino]- 9H-purin-2-yl]amino]-5- thiazolecarboxylic acid ethyl ester | 2.56 | 579.30 |
| A7 | 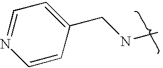 | 4-Methyl-2-[[9-[[4- (methylsulfonyl)phenyl]methyl]- 6-[(4-pyridinylmethyl)amino]- 9H-purin-2-yl]amino]-5- thiazolecarboxylic acid ethyl ester | 2.60 | 579.28 |

^{a}HPLC conditions used to determine retention times; 4 min gradient 0-100% B in A(A; 0.1% TFA in 90/10 water/methanol; B; 0.1% TFA in 10/90 water/methanol) using a YMC turbopack column at 254 nm.

### Example A8

### 2-[[6-[[(3,4-Dimethoxyphenyl)methyl]amino]-9-ethyl-9H-purin-2-yl]amino]-4-methyl-5-thiazolecarboxylic Acid, Ethyl Ester, Trifluoroacetate (1:1).

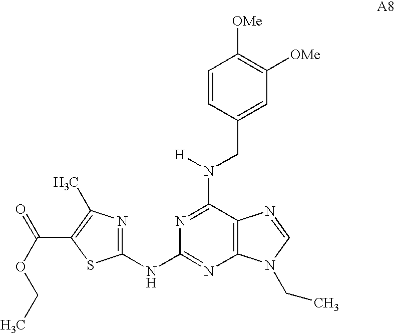

A8.1: Preparation of N-7-ethyl-2,6-dichloropurine and N-9-ethyl-2,6-dichloropurine

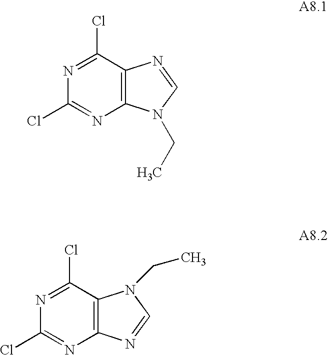

2,6-Dichloropurine (5.0 g, 26.7 mmol), potassium carbonate (11.1 g, 80 mmol) and, ethyl iodide (6.4 ml, 80 mmol) were refluxed in acetone (250 ml) for 2-3 h until tic (30% ethyl acetate in dichloromethane) showed no more starting material. The mixture was cooled, filtered and concentrated to give a 3:1 mixture of N-9:N-7 alkylated purine as determined by HPLC. The products were purified by chromatography over silica gel (5% ethyl acetate in dichloromethane->40% ethyl acetate in dichloromethane) to give N-9-ethyl-2,6-dichloropurine (A2.1) (3.71 g, 64.2% yield) and N-7-ethyl-2,6-dichloropurine (A2.2) (0.943g, 16.3% yield).

A8.2: 2-[[6-[[(3,4-Dimethoxyphenyl)methyl]amino]-9-ethyl-9H-purin-2-yl]amino]-4-methyl-5-thiazolecarboxylic Acid, Ethyl Ester, Trifluoroacetate (1:1).

The dichloropurine A8.1 was reacted in a manner similar to step A1.2 substituting 3,4-dimethoxybenzylamine for methylamine to produce an intermediate monochloropurine which was reacted in a manner essentially identical to step A1.3 to produce A8.

### Example A9

### 2-[[9-[(3,4-Dimethoxyphenyl)methyl]-6-(4-morpholinyl)-9H-purin-2-yl]amino]-4-methyl-5-thiazolecarboxylic Acid, Ethyl Ester

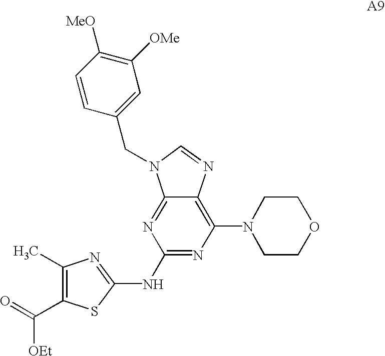

Example A9 was prepared in a manner analogous to Example A1 with the exceptions that in step A1.1, 4-methylsulfonylbenzyl chloride was substituted with 3,4-dimethoxybenzyl chloride, and in step A1.2, methylamine was replaced with morpholine. Step A1.3 was conducted in an almost identical manner substituting the appropriate monochloropurine. LCMS: Ret. Time=3.59 min, M+=498.13. HPLC conditions used to determine retention times; 4 min gradient 0-100% B in A(A; 0.1% TFA in 90/10 water/methanol; B; 0.1%TFA in 10/90 water/methanol) using a YMC turbopack column at 254 nm

### Example A10

### 2-[[9-[(pyridin-3-yl)methyl]-6-(4-morpholinyl)-9H-purin-2-yl]amino]-4-methyl-5-thiazolecarboxylic Acid, Ethyl Ester

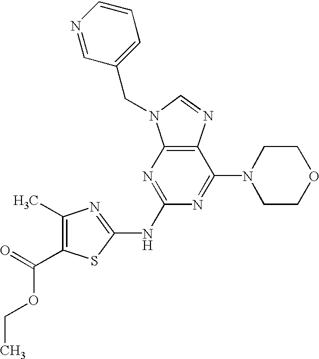

A10

Example A10 was prepared in a manner analogous to Example A9 with the exceptions that in step A1.1, 4-methylsulfonylbenzyl chloride was substituted for 3-picolylchloride hydrochloride. LCMS: retention time=1.54 min, M+=480.00. HPLC conditions used to determine retention times; 2 min gradient 0-100% B in A(A; 0.1% TFA in 90/10 water/methanol; B; 0.1% TFA in 10/90 water/methanol) using a Phenomenex® column at 220 nm detection.

### Example A11

N-[4-(5-Methyl-2-pyrimidinyl)phenyl]-6-(4-morpholinyl)-9-(2-pyridinylmethyl)-9H-purin-2-amine

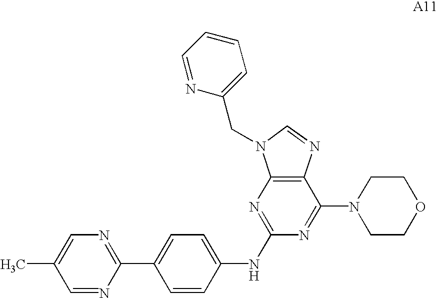

Example A11 was prepared in a manner analogous to Example A1 with the exceptions that in step A1.1, 4-methylsulfonylbenzyl chloride was substituted with 2-picolylchloride hydrochloride, and in step A1.2, methylamine was replaced with morpholine. Step A1.3 was conducted in an almost identical manner substituting A11.1, 4-(4-methylpyrimidn-2-yl)aniline for ethyl-2-amino-4-methylthiazole-5-carboxylate. LCMS: retention time=1.51 min, M+=479.00. HPLC conditions used to determine retention times; 2 min gradient 0-100% B in A(A; 0.1% TFA in 90/10 water/methanol; B; 0.1% TFA in 10/90 water/methanol) using a Phenomenex® column at 220 nm detection.

A11.1: 4-(4-methylpyrimidn2-yl)aniline for ethyl-2-amino-4-methylthiazole-5-carboxylate

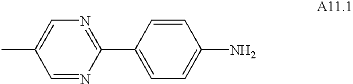

4-Aminobenzamidine dihydrochloride (2.1 g., 0.01 mmol), and 3-ethoxymethacreolein (1.2 g, 0.010 mmol) were dissolved in methanol at room temperature. 25% Sodium methoxide (4.3 g, 0.020 mmol) was added and the reaction mixture stirred for 1.5 h. The solvent was evaporated under reduced pressure, and the resulting oil partitioned between water and ether. The organic layer was dried with magnesium sulfate, filtered and evaporated to provide 1.2 g (65% yield) A11.1 as a solid. MS (M+H)⁺=185.

### Example B1

### Example B1

### 2-[[4-[[[4-(Aminosulfonyl)phenyl]methyl]amino]-6-chloro-2-pyrimidinyl]amino]-4-4-methyl-5-thiazolecarboxylic Acid Ethyl Ester

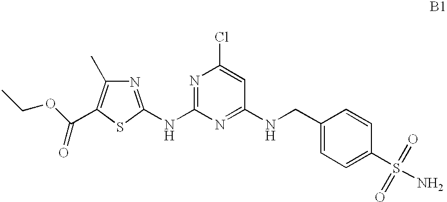

B1.1: 2-[(Aminoiminomethyl)amino]-4-methyl-5-thiazolecarboxylic Acid Ethyl Ester

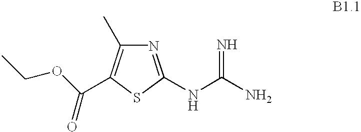

A solution of 2-imino-4-thiobiuret (20.0 g, 0.17 mol) 2-chloroacetoacetate (28 g, 0.17 mol) in ethanol (500 mL) was heated to 100° C. for 4 hours. The reaction mixture was concentrated to half volume and poured into 1 liter of 1N NaOH. The white solid which precipitated out was collected by filtration and dried under vacuum to yield B1.1 (30.5 g, 79%). ¹H-NMR (DMSO-d₆) δ: 4.22 (2H, q, J=7 Hz ), 2.50 (3H, merge with DMSO ), 1.26 (3H, t, J=7 Hz). HPLC: 97.7%, ret. time=1,619 min., LC/MS (M+H)⁺=229.

B1.2: 2-[(4-6(1H,5 H)-pyrimidinedion-2-yl)amino]-4-methyl-5-thiazolecarboxylic Acid Ethyl Ester

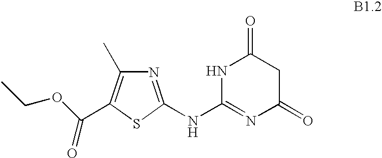

To a solution of B1.1 (5.7 g, 25 mmol) in ethanol (250 mL) was added 21% sodium ethoxide in ethanol (7.75 mL, 25 mmol). The reaction mixture was heated in an oil bath at 100° C. for 15 minutes during which time most, but not all, of the material had dissolved, and Diethylmalonate (3.8 g, 25 mmol) was added. The reaction mixture was maintained in an oil bath to 100° C. for 2 hours. An additional 4 mL of 21% sodium ethoxide in ethanol and additional 2 mL of diethylmalonate were added and the reaction mixture refluxed for an additional 2 hours after which HPLC analysis indicated only a trace amount of starting material remained. The reaction mixture was allowed to cool to room temperature and the copious crystals which precipitated out were collected by filtration and dried to yield B1.2 solvated with 1 molecule of ethanol (7.6 g, 89% based on solvate). ¹H-NMR (DMSO-d₆) δ: 9.75 (1H, brs) 4.45 (1H, t, J=4 Hz), 4.14 (2H, q, J=7 Hz), 3.45 (2H, m) 2.56 (3H, s), 1.29 (3H, t, J=7 Hz). 1.05 (3H, t, J=7 Hz), HPLC: 91.5%, ret. time=2.836 min., LC/MS (M+H)⁺=297.

B1.3: 2-[(4-6-Dichloropyrimidin-2-yl)amino]-4-methyl-5-thiazolecarboxylic Acid Ethyl Ester

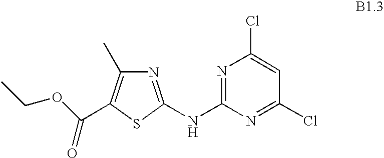

A suspension of B1.2 (7.6 g, 22 mmol) in POCl₃ (54 ml) was heated at 100° C. for 16 hours and then it was cooled down to RT which was poured into 500 g of ice. After the ice melted the solid was collected by filtration and triturated with hot methanol. The solid was then dried under vacuum to yield. B1.3 (6.2 g, 84%). 1H-NMR (DMSO-d₆) δ: 7.55 (1H, s ), 4.27 (2H, q, J=7 Hz), 2.56 (3H, s), 1.29 (3 H, t, J=7 Hz). HPLC: 97%, ret. time=3.929 min., LC/MS (M+H)⁺=333.

B1.4: 2-[[4-[[[4-(Aminosulfonyl)phenyl]methyl]amino]-6-chloro-2-pyrimidinyl]amino]-4-methyl-5-thiazolecarboxylic Acid Ethyl Ester

A suspension solution of B1.3 (33 mg, 0.1 mmol), p-aminomethyl-benzenesulfonamide•HCl (24 mg, 0.106 mmol) and diisopropylethylamine (58 mg, 0.45 mmol) in n-butanol (2 mL) was heated to 105° C. for 2 hours and then it was cooled down to RT. The solid was precipitated out which was collected with filtration to yield B1 (31.8 mg, 66%). ¹H-NMR (DMSO-d₆) δ: 7.77 (2H, d, J=8 Hz), 7.52 (2H, d, J=8 Hz), 7.31 (2H, s), 6.27 (1H, s), 4.81 (2H, m), 4.22 (2H, q, J=7 Hz), 2.50 (3H, merge with DMSO), 1.26 (3H, t, J=7 Hz ). HPLC: 96%, ret. time=3.232 min., LC/MS (M+H)⁺=483.

### Example B2

### 2-[[4-[[[4-(Aminosulfonyl)phenyl]methyl]amino]-6-(methylamino)-2-pyrimidinyl]amino]-4-methyl-5-thiazolecarboxylic Acid, Ethyl Ester

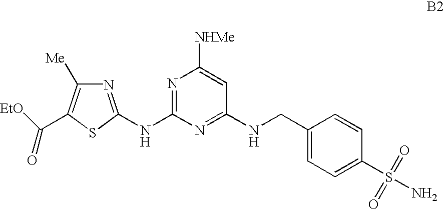

B2.1: 2-[[4-[[[4-(Aminosulfonyl)phenyl]methyl]amino]-6-(methylamino)-2-pyrimidinyl]amino]-4-methyl-5-thiazolecarboxylic Acid, Ethyl Ester

Methylamine hydrochloride, (0.14 g, 2.0 mmol), and B1 (0.39 g, 0.81 mmol), were dissolved in 1-methyl-2-pyrrolidinone (3 mL) and placed in a sealed tube reaction vessel. Diisopropylethylamine (0.78 g, 6.0 mmol) was added, the vessel sealed and the reaction mixture was heated at 130° C. for approximately 24 h. The vessel was cooled below room temperature in and ice bath and cautiously opened. The crude product was collected by filtration. Trituration of this material with a copious amount (approximately 100 mL) of methanol for 1 h followed by filtration provided 333 mg (81%) of B2 as an off-white solid. ¹H-NMR (DMSO-d₆) δ: 7.74 (2H, d, J=8 Hz), 7.49 (2H, d, J=8 Hz), 7.27 (2H, s), 6.27 (1H, s), 4.81 (2H, m), 4.22 (2H, q, J=7 Hz), 2.50 (3H, merge with DMSO), 1.26 (3H, t, J=7 Hz). HPLC: 96%, ret. time=3.232 min., LC/MS (M+H)⁺=483.

### Example B3-B8

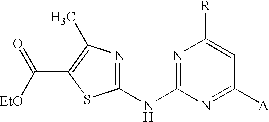

Examples B3 to B8 were prepared in a similar manner to that used for Example B1 or B2 utilizing the appropriate amines.

TABLE B

| | | | | HPLC | |
|---|---|---|---|---|---|
| | | | | Retention^{a} | MS |
| Ex. | R | A | Name | (min) | Reported |
| B3 | 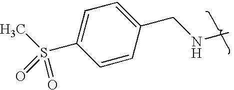 | Cl | 2-[[4-Chloro-6-[[[4- (methylsulfonyl)phenyl]methyl]amino]-2- pyrimidinyl]amino]-4- methyl-5- thiazolecarboxylic acid, ethyl ester | 1.43 | 482.21 |
| B4 | 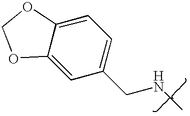 | 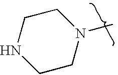 | 2-[[4-[(1,3- Benzodioxol-5- ylmethyl)amino]-6-(1- piperazinyl)-2- pyrimidinyl]amino]-4- methyl-5- thiazolecarboxylic acid ethyl ester | 2.13 | 498.48 |
| B5 | 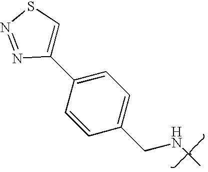 | 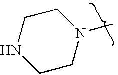 | 4-Methyl-2-[[4-(1- piperazinyl)-6-[[[4- (1,2,3-thiadiazol-4- yl)phenyl]methyl]amino ]-2-pyrimidinyl]amino]- 5-thiazolecarboxylic acid ethyl ester | 2.18 | 538.42 |
| B6 | 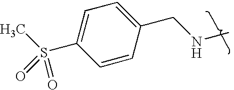 | 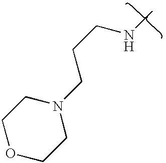 | 4-Methyl-2-[[4-[[[4- (methylsulfonyl)phenyl]methyl]amino]-6-[[3-(4- morpholinyl)propyl]ami no]-2- pyrimidinyl]amino]-5- thiazolecarboxylic acid ethyl ester | 1.13 | 590.37 |
| B7 | 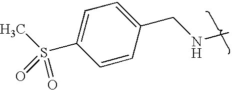 | 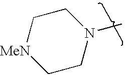 | 4-Methyl-2-[[4-(4- methyl-1-piperazinyl)- (methylsulfonyl)phenyl]methyl]amino]-2- pyrimidinyl]amino]-5- thiazolecarboxylic acid ethyl ester | 1.28* | 546.18 |
| B8 | 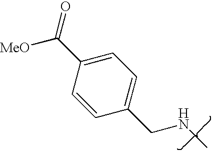 | 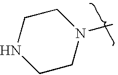 | 2-[[4-[[[4- (Methoxycarbonyl)phen yl]methyl]amino]-6-(1- piperazinyl)-2- pyrimidinyl]amino]-4- methyl-5- thiazolecarboxylic acid ethyl ester | 1.53 | 512.17 |

^{a}HPLC conditions used to determine retention times; 4 min gradient 0-100% B in A(A; 0.1% TFA in 90/10 water/methanol; B; 0.1% TFA in 10/90 water/methanol) using a YMC turbopack column at 254 nm.
* Waters Xterra 4.6 × 30 5u C18 (2 min) Solvent A and B as above.

### Example B9

### 1 -Acetyl-5-{4-(4-methyl-piperazin-1-yl)-6-[[[4-(aminosulfonyl)phenyl]methyl]amino]pyrimidin-2-ylamino}-2,3-dihydro-1 H-tetrahyroindole

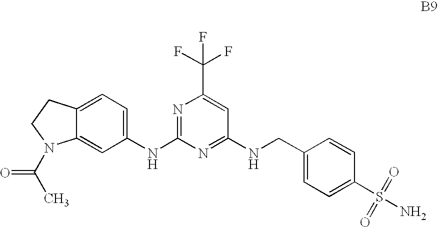

B9.1: 4-Chloro-2-methylthio-6-trifluoromethylpyrimidine

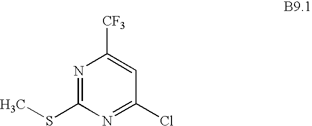

A mixture of commercially available 4-hydroxy-2-methylthio-6-trifluoromethylpyrimidine (2.00 g, 9.52 mmol) and POCl₃ (10 mL) was heated at reflux for 1.5 h. The excess POCl₃ was removed under vacuum. The residue was dissolved in AcOEt, washed with cold water, saturated NaHCO₃ solution, cold water, and brine. The solution was then dried over anhydrous MgSO₄. Evaporation of solvent provided B9.1 (1.31 g, 60% yield) as a colorless oil.

B9.2: 4-[[[4-(Aminosulfonyl)phenyl]methyl]amino]-2-methylthio-6-trifluoromethylpyrimidine

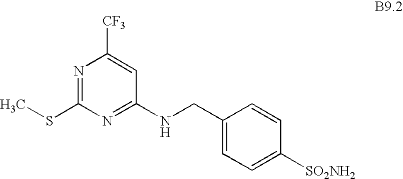

A mixture of B9.1 (1.28 g, 5.60 mmol), 4-aminomethylbenzenesulfonamide hydrochloride (1.97 g, 8.85 mmol), and triethylamine (1.76 mL, 12.6 mmol) in ethanol (15 mL) was heated at 85° C. in a sealed tube for 1 h. The mixture was concentrated under vacuum. The residue was diluted with AcOEt, washed with water, 1N AcOH (twice), saturated NaHCO₃ solution (twice), and brine. The solution was then dried over anhydrous MgSO₄. Evaporation of solvent provided B9.2 (2.10 g, 99% yield) as a white solid.

B9.3: 4-[[[4-(Aminosulfonyl)phenyl]methyl]amino]-2-methylsulfonyl-6-trifluoromethylpyrimidine

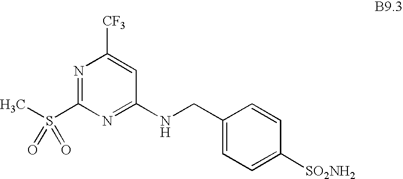

To a solution of B9.2 (1.88 g, 4.97 mmol) in MeOH (130 mL) was added mCPBA (75%, 3.42 g, 14.9 mmol) at rt in one portion. The resulting mixture was stirred at rt for 16 h before it was concentrated under vacuum. The residue was diluted with AcOEt, washed with 5% NaS₂O₃ solution (twice), saturated NaHCO₃ solution (twice), and brine. The solution was then dried over anhydrous MgSO₄. Evaporation of solvent provided B9.3 (2.00 g, 98% yield) as a white solid.

B9.4: 1-Acetyl-5-{4-(4-methyl-piperazin-1-yl)-6-[[[4(aminosulfonyl)phenyl]methyl]amino]pyrimidin-2-ylamino}-2,3-dihydro-1H-tetrahyroindole

A mixture of B9.3 (20 mg, 0.048 mmol) and commercially available 1-Acetyl-5-amino-2,3-dihydro-(1H)indole (84 mg, 0.48 mmol) was fused at 175° C. for 20 min. After cooling to rt, the mixture was dissolved in a minimum amount of DMSO, diluted with MeOH, and applied to preparative HPLC. B9 (16 mg, 46% yield) was obtained as a lyophilized powder as a 2 eq. TFA salt. (M+H )⁺=507.09.

### Example C1

### ′2-[[4-[[[4-(Methylsulfonyl)phenyl]methyl]amino]-5,6,7,8-tetrahydro-6-methylpyrido[4,3-d]pyrimidin-2-yl]amino]-4-methyl-5-thiazolecarboxylic Acid Ethyl Ester

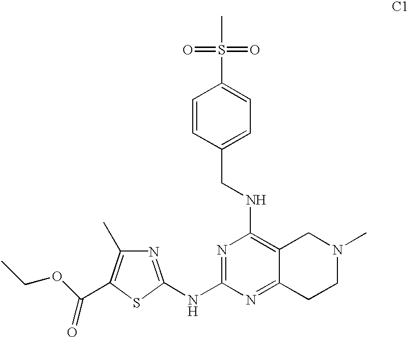

C1.1: N-(3-Methoxy-3-oxopropyl)-N-methyl-β-alanine Methyl Ester

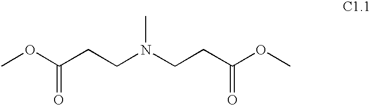

A solution of methyl acrylate (3.79 g, 44 mmol) and methyl amine (2M in methanol, 10 ml, 20 mmol) was heated to 100° C. in a sealed pressure tube for 2 days. The reaction mixture was concentrated to give a crude product which was purified on silica gel column with dichloromethane/methanol (50/1). The fractions which contained the product was concentrated and dried over vacuum pump to yield C1.1 (3.96 g, 86%). ¹H-NMR (CDCl₃) δ: 3.70 (6H, s), 2.74 (4H, t, J=7 Hz), 2.50 (4H, t, J=7 Hz), 2.27 (3H, s).

C1.2: 1-Methyl-4-oxo-3-piperidinecarboxylic Acid Methyl Ester

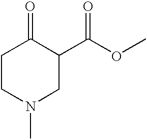

To a solution of sodium methoxide (25% in methanol, 4.74 ml, 20 mmol) in toluene (40 ml) at 110° C. was added C1.1 (2.0 g, 9.84 mmol). The reaction mixture was refluxed for 1 hr and then it was cooled down to room temperature. The reaction mixture was concentrated to give a crude product which was purified on silica gel column with dichloromethane/methanol (20/1). The fractions which contained the product was concentrated and dried over vacuum pump to yield the desired product C1.2 (1.61 g, 96%). ¹H-NMR ( CD₃OD) δ: 3.50 (3H, s), 3.25 (1H, m), 3.09 (1H, m), 2.60-2.70 (1H, m), 2.44-2.51 (1H, m), 2.14-2.34 (5H, m). HPLC: 96%, ret. time=0.18 min., LC/MS (M+H)⁺=172

C1.3: 2-(4-Methyl-5-ethoxycarbonylthiazol-2-ylamino)-5,6,7,8-tetrahydro-6-methyl pyrido[4,3-d]pyrimidin-4-ol

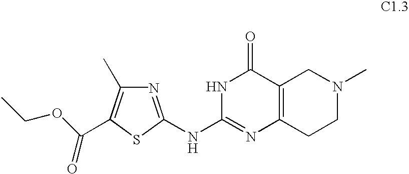

A solution of C1.2 (125 mg, 0.731 mmol), B1.1 (167 mg, 0.731 mmol) and sodium ethoxide(21% in ethanol, 0.989 ml, 2.65 mmol) in DMA was heated to 100° C. for 1 hr and then it was cooled down to RT. The reaction mixture was diluted with 2 mL of water, and neutralized with 1 N HCl. The solid was collected by filtration and dried to yield B1.3 (150 mg, 59%).

C1.4: 2-(4-Methyl-5-ethoxycarbonylthiazol-2-ylamino), 4-chloro-5,6,7,8-tetrahydro-6-methyl-pyrido[4.3-d]pyrimidine

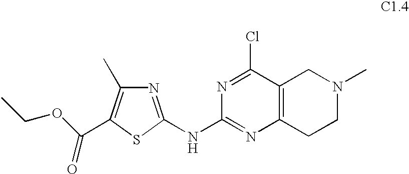

A solution of C1.3 (150 mg, 0.429 mmol) in POCl₃ (1 ml) was heated to 100° C. for 2 hours and then it was cooled down to RT which was poured into 10 ml of ice-water. It was neutralized with NaOH to pH about 9. The solid was collected with filtration and then it was added to 10 ml of methanol and stirred about 10 minutes. The solid was filtered off. The mother solution was concentrated to yield the desired product C1.4 (70 mg, 44.3%). LC/MS (M+H)⁺=368.

C1.5: ′2-[[4-[[[4-(Methylsulfonylphenyl]methyl]amino]-5,6,7,8-tetrahydro-6-methylpyrido[4,3-d]pyrimidin-2-yl]amino]-4-methyl-5-thiazolecarboxylic Acid Ethyl Ester

A solution of C1.4 (70 mg, 0.19 mmol) and 4-methylsulfonylbenzylamine hydrochloric salt (66 mg, 0.285 mmol), diisopropylethylamine (111 mg, 0.855 mmol) in N-methyl-2-pyrrolidine (2 mL) was heated to 120 to 130° C. for two hours. The reaction mixture was concentrated to yield a crude product which was purified with prep. HPLC (reverse phase) to yield C1 (38 mg, 32%). ¹H-NMR (CD₃OD) δ: 7.78 (2H, d, J=8 Hz), 7.52 (2H, d, J=8Hz ) 4.92 (2H, s), 4.17 (2H, q, JJ=7 Hz), 4.03 (2H, m), 3.45 (2H, m), 2.93-2.98 (8H, m), 2.40 (3H, s), 1.18 (3H, t, J=7 Hz). HPLC: 98%, ret. time=1.58 min., LC/MS (M+H)⁺=517.

### Example C2

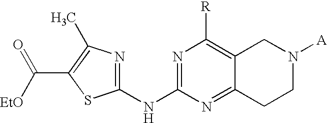

Examples C2-was prepared in a similar manner to that used for Example C1.

TABLE C

| | | | | HPLC | |
|---|---|---|---|---|---|
| | | | | Retention | MS |
| Ex. | R | A | Name | (min) | Reported |
| C2 | 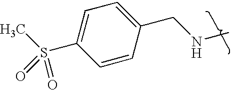 | Me | 2-[[4-[[[4- (Aminosulfonyl)phenyl]meth yl]amino]-5,6,7,8-tetrahydro- 6-methylpyrido[4,3- d]pyrimidin-2-yl]amino]-4- methyl-5-thiazolecarboxylic acid ethyl ester | 1.467 | 518.12 |

### Example D1

### 2-[[7-[(Acetyloxy)acetyl]-6,7,8,9-tetrahydro-4-[[[4-(methylsulfonyl)phenyl]methyl]amino]-5H-pyrimido[4,5-d]azepin-2-yl]amino]-4-methyl-5-thiazolecarboxylic Acid Ethyl Ester

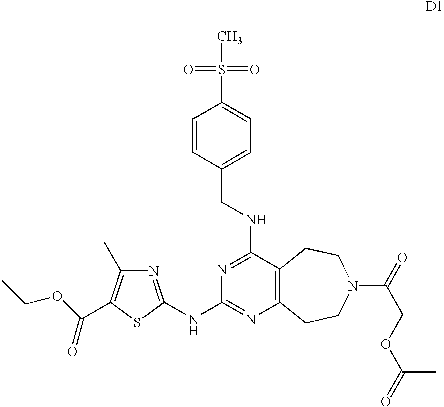

### D1.1: Hexahydro-5-oxo-1H-Azepine-1,4-dicarboxylic Acid 4-tertbutyl 1-methyl Ester

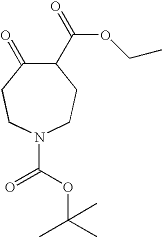

A solution of commercially available N-tertbutoxycarbonyl-4-piperidone (500 mg, 2.46 mmol) in 2 mL of ethyl ether (2 mL) was simultaneously added boron trifluoride etherate (349 mg, 2.46 mmol) and ethyl diazoacetate dropwise (371 mg, 3.25 mmol) at −25° C. to −30° C. The reaction mixture was maintained at −25° C. to −30° C. for one hour and then it was warmed to RT. The reaction mixture was diluted with ethyl ether (30 ml) and was washed with saturated Na₂CO₃ solution (20 mL) and the organic layer dried over sodium sulfate. Filtration and concentration to yield a crude product which was purified on silica gel column with dichloromethane/methanol (50/1 to 20/1) to yield D1.1 (662 mg, 94.4% ). HPLC: 91%, retention time: 3.677 minute.

D1.2: 2-(4-Methyl-5-ethoxycarbonylthiazol-2-ylamino)-5,6,8,9-tetrahydro-7-tertbutyloxycarbonylpyrido[4,5-d]azepin-4-ol

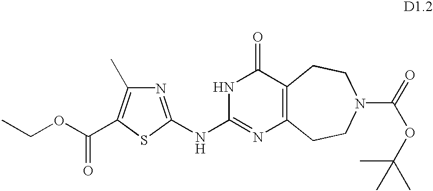

A solution of B1.1 (110 mg, 0.485 mmol) and sodium ethoxide (21% in ethanol, 0.656 ml, 1.76 mmol) in ethanol (2 ml) was heated to 100° C. for half an hour and then it was cooled down to RT which was added D1.1 (138 mg, 0.485 mmol). The reaction mixture was heated to 100° C. for 2 days. It was concentrated to yield a crude product which was diluted with 2 mL of water and neutralized with 1 N HCl. The solid was collected by filtration and stirred with anhydrous methanol for 10 minutes. The resulting solid was collected by filtration to yield D1.2 (77 mg, 35%). LC/MS (M+H)⁺=450.35.

D1.3: 4-Chloro-2-(4-methyl-5-ethoxycarbonylthiazol-2-ylamino)-5,6,8,9-tetrahydro-7H-pyrido[4,5-d]azepine

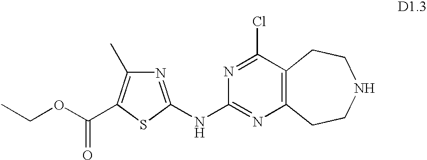

A solution of D1.2 (77 mg, 0.172 mmol) in POCl₃ (0.5 ml) was heated to 100° C. for 16 hours and then it was cooled down to RT which was poured into 5 ml of ice-water. It was neutralized with NaOH to pH about 9. The solid was collected by filtration and then it was added to 3 mL of methanol and stirred about 20 minutes. The solid was collected to yield D1.3 (67 mg). LC/MS (M+H)⁺=368.11. HPLC:>98%, retention time: 2.390 min.

D1.4: 2-[[7-[(Acetyloxy)acetyl]-6,7,8,9-tetrahydro-4-chloro-5H-pyrimido[4,5-d]azepin-2-yl]amino]-4-methyl-5-thiazolecarboxylic Acid Ethyl Ester

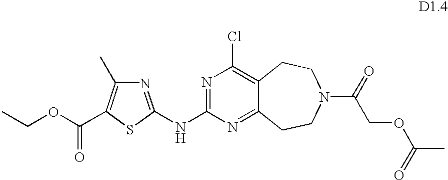

A solution of D1.3 (120 mg, 0.326 mmol) & pyridine (38.7 mg, 0.489 mmol) in N,N-dimethylformamide (1.5 ml) was added acetoxyacetyl chloride (55 mg, 0.391 mmol) at 0-5° C. The reaction mixture was warmed up to RT and stirred for 1.5 hrs, then was heated to 90° C. for 1 hr after which time the reaction had not proceeded to a significant extent. The reaction mixture was cooled down to RT and diisopropylethylamine (105 mg, 0.815 mmol) was added at RT, followed by acetoxyacetyl chloride (110 mg, 0.782 mmol). The reaction mixture was stirred at RT for 1 hr and diisopropylethylamine (105 mg, 0.815 mmol) was added at RT, followed by acetoxyacetyl chloride (110 mg, 0.782 mmol). After stirred at RT for 1 hr, the reaction mixture was concentrated to yield a crude product which was added water (5 ml) and stirred for 5 minutes. The solid was collected with filtration to yield D1.4 (65 mg, 43% ), LC/MS (M+H)+=468.42.

D1.5: 2-[[7-[(Acetyloxy)acetyl]-6,7,8,9-tetrahydro-4-[[[4-(methylsulfonyl)phenyl]methyl]amino]-5H-pyrimido[4,5-d]azepin-2-yl]amino]-4-methyl-5-thiazolecarboxylic Acid Ethyl Ester

A solution of D1.4 (65 mg, 0.139 mmol), 4-methylsulfonylbenzylamine•HCl (66 mg, 0.285 mmol) and diisopropylethylamine (111 mg, 0.855 mmol) in N-methyl-2-pyrrolidine (2 ml) was heated to 120° C. for 2 hrs and then it was cooled down to RT. The reaction mixture was concentrated to yield a crude product which was added MeOH (20 ml) and stirred for 20 minutes. Filtration to remove the solid and concentration to yield the crude product which was purified with prep HPLC to yield D1 (16 mg, 19%). ¹H-NMR (CD₃OD) δ: 7.96 (2H, d, J=8 Hz), 7.65-7.72 (2H, m), 5.16 (2H, d, J=6 Hz ), 4.36 (2H, m), 3.76-4.00 (44H, m), 3.25 (1H, m), 2.89-3.20(8H, m), 2.60 (3H, s), 2.18 (3H, d, J=5Hz), 1.38 (3H, m). HPLC: 87%, ret. time=2.303 min., LC/MS (M+H)⁺=617.15.

### Example D2

### 4-Methyl-2-[[6,7,8,9-tetrahydro-7-(hydroxyacetyl)-4-[[[4-(methylsulfonyl)phenyl]methyl]amino]-5H-pyrimido[4,5-d]azepin-2-yl]amino]-5-thiazolecarboxylic Acid Ethyl Ester

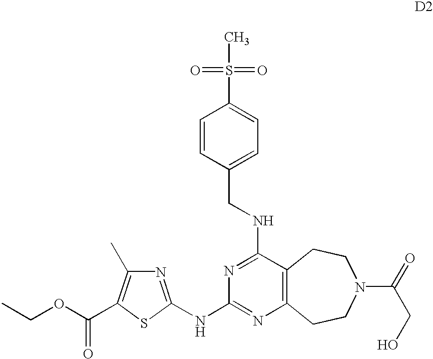

A solution of D1 (15 mg, 0.0244 mmol) and ammonium hydroxide (5 drops ) in methanol (1 ml) was stirred at RT for 5 hrs. The reaction mixture was concentrated to yield D2(12.7 mg, 91%). ¹H-NMR (CD₃OD) δ: 7.91 (2H, d, J=8 Hz), 7.60-7.68 (2H, m), 5.10 (2H, d, J=6 Hz ), 4.26-4.36 (4H, m), 3.82-3.95 (2H, m), 3.03-3.20 (6H, m), 2.88-3.00 (3H, m), 2.52 (3H, s ), 1.27-1.38 (3H, m). HPLC: 85% . ret. time=2.190 min., LC/MS (M+H)⁺=575.13.

### Example E1

### 2-[[4-[[[4-(Aminosulfonyl)phenyl]methyl]amino]-2-quinazolinyl]amino]-4-methyl-5-thiazolecarboxylic Acid Ethyl Ester

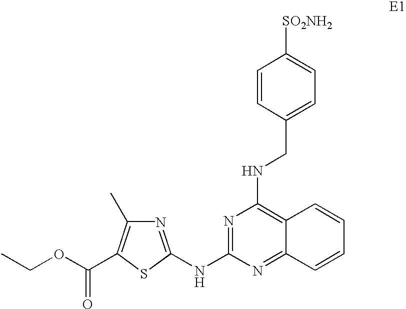

### E1.1: 2-Chloro-4-(4-methylsulfonylbenzyl)quinazoline

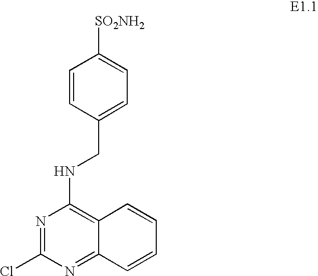

A mixture of 2,4-dichloroquinazoline [prepared from benzoyleneurea and POCl₃ by the method of Butler et al., J. Chem. Soc. 1959, 1512.) (100 mg, 0.502 mmol, 1 eq) , 4-aminosulfonylbenzylamine hydrochloride (117.5 mg, 0.527 mmol, 1.05 eq) and diisopropylethylamine (0.26 mL, 1.506 mmol, 3 eq) in absolute ethanol (1.6 mL) was stirred at ambient temperature for 4 h. The precipitated solid was collected by filtration, washed with water and cold ethanol, and dried to afford 154 mg (88%) of 2-chloro-4-(4-aminosulfonylbenzyl)quinazoline as a white solid. LC/MS: 349 [M+H]⁺; HPLC: 96% at 1.86 min (Primesphere 5 μm C18 column 4.6×30 mm, 10-90% aqueous methanol over 2 min containing 0.2% phosphoric acid, 5 mL/min, monitoring at 254 nm); ¹H NMR (400 MHz, DMSO-d₆): δ9.37 (t, J=5.8 Hz, 1 H), 8.32 (d, J=8.2 Hz, 1 H), 7.85-7.53 (m, 7 H), 7.32 (s, 2 H), 4.81 (d, J=5.7 Hz, 2 H).

E1.2: 2-[[4-[[[4-(Aminosulfonyl)phenyl]methyl]amino]-2-quinazolinyl]amino]-4-methyl-5-thiazolecarboxylic Acid Ethyl Ester

To a mixture of El.1 (77 mg, 0.221 mmol, 1 eq) and ethyl 2-amino-4-methylthiazole-5-carboxylate (82 mg, 0.442 mmol, 2 eq) in N,N-dimethylacetamide (2.2 mL) in a 2-dram vial was added tris(dibenzylideneacetone)dipalladium(0) (20.2 mg, 0.022 mmol, 0.1 eq), 2-(di-t-butylphosphino)biphenyl (19.8 mg, 0.066 mmol, 0.3 eq) and sodium t-butoxide (42.5 mg, 0.442 mmol, 2 eq). The vial was purged with N₂, sealed and heated in a 105° C. oil bath for 2.25 h. The reaction mixture was cooled to rt, filtered and concentrated in vacuo. The residue was treated with methanol (ca. 1 mL) and the precipitated solid was collected by filtration, washed with methanol and dried to afford 41 mg (37%) of product E1 as a tan solid. LC/MS: 499 [M+H]⁺; HPLC: >95% at 1.92 min (Primesphere 5 μm C18 column 4.6×30 mm, 10-90% aqueous methanol over 2 min containing 0.2% phosphoric acid, 5 mL/min, monitoring at 254 nm); ¹H NMR (400 MHz, DMSO-d₆): δ11.55 (br s, 1 H), 9.12 (br s, 1 H), 8.23 (d, J=8.2 Hz, 1 H), 7.77-7.54 (m, 6 H), 7.36 (t, J7.5 Hz, 1 H), 7.28 (br s, 2 H), 4.93 (br s, 2 H), 4.24 (q, J=7.1 Hz, 2 H), 2.50 (coincident with residual DMSO, 3 H), 1.29 (t, J=7.1 Hz, 3 H).

### Example F1

### 2-[[4-[4-(Dimethylamino)-1-piperidinyl]-6-[[(3,4,5-trimethoxphenyl)methyl]amino]-2-pyrimidinyl]amino]-4-methyl-5-thiazolecarboxylic Acid, Ethyl Ester

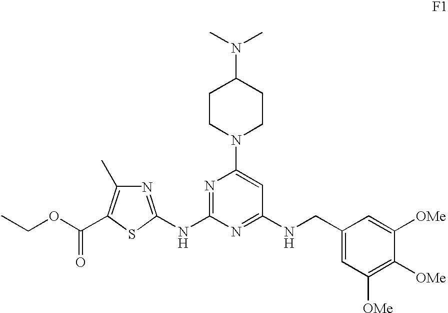

F1.1: 2-[(Aminoiminomethyl)amino]-4-methyl-5-thiazolecarboxylic Acid Ethyl Ester

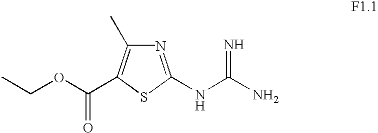

A solution of 2-imino-4-thiobiuret (20.0 g, 0.17 mol), 2-chloroacetoacetate (28 g, 0.17 mol) in ethanol (500 mL) was heated to 100° C. for 4 hours. The reaction mixture was concentrated to half volume and poured into 1 liter of 1N NaOH. The white solid which precipitated out was collected by filtration and dried under vacuum to yield F1.1 (30.5 g, 79%). ¹H-NMR ( DMSO-d₆) δ: 4.22 (2H, q, J=7 Hz ), 2.50 (3H, merge with DMSO ), 1.26 (3H, t, J=7 Hz). HPLC: 97.7%, ret. time=1.619 min., LC/MS (M+H)⁺=229.

F1.2: 2-[(4-6(1H,5H)-pyrimidinedion-2-yl)amino]-4-methyl-5-thiazolecarboxylic Acid Ethyl Ester

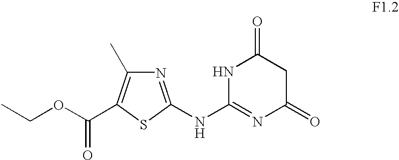

To a solution of F1.1 (5.7 g, 25 mmol) in ethanol (250 mL) was added 21% sodium ethoxide in ethanol (7.75 mL, 25 mmol). The reaction mixture was heated in an oil bath at 100° C. for 15 minutes during which time most, but not all, of the material had dissolved, and Diethylmalonate (3.8 g, 25 mmol) was added. The reaction mixture was maintained in an oil bath to 100° C. for 2 hours. An additional 4 mL of 21% sodium ethoxide in ethanol and additional 2 mL of diethylmalonate were added and the reaction mixture refluxed for an additional 2 hours after which HPLC analysis indicated only a trace amount of starting material remained. The reaction mixture was allowed to cool to room temperature and the copious crystals which precipitated out were collected by filtration and dried to yield F1.2 solvated with 1 molecule of ethanol (7.6 g, 89% based on solvate ). ¹H-NMR ( DMSO-d₆) δ: 9.75 (1H, br s) 4.45 (1H, t, J=4 Hz), 4.14 (2H, q, J=7 Hz), 3.45 (2H, m) 2.56 (3H, s), 1.29 (3H, t, J=7 Hz ). 1.05 (3H, t, J=7 Hz), HPLC: 91.5%, ret. time=2.836 min., LC/MS (M+H)⁺=297.

F1.3: 2-[(4-6-Dichloropyrimidin-2-yl)amino]-4-methyl-5-thiazolecarboxylic Acid Ethyl Ester

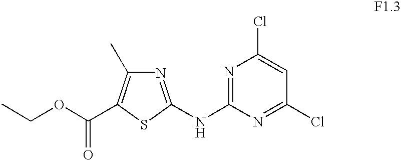

A suspension of F1.2 (7.6 g, 22 mmol) in POCl₃ (54 ml) was heated at 100° C. for 16 hours and then it was cooled down to RT which was poured into 500 g of ice. After the ice melted the solid was collected by filtration and triturated with hot methanol. The solid was then dried under vacuum to yield. F1.3 (6.2 g, 84%).¹H-NMR (DMSO-d₆) δ: 7.55 (1H, s ), 4.27 (2H, q, J=7 Hz ), 2.56 (3H, s ), 1.29 (3H, t, J=7 Hz). HPLC: 97%, ret. time=3.929 min., LC/MS (M+H)⁺=333.

F1.4: 2-[[4-[4-(Dimethylamino)-1-piperidinyl]-6-chloro-2-pyrimidinyl]amino]-4-methyl-5-thiazolecarboxylic Acid, Ethyl Ester

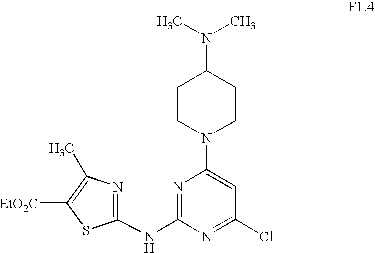

A suspension of dichloropyrimidine F1.3 (1.0 g, 3.0 mmol), 4-dimethylamino piperidine (0.42 g, 3.3 mmol) and diisopropylethylamine (2.3 ml, 13.2 mmol) in n-butanol (20 ml) was heated to 105° C. for 3 hours. After cooling to room temperature, the precipitated solid was collected by filtration and washed with methanol to yield F1.4 (1.1 g, 84%). HPLC: 95%, ret. time=3.320 min., LC/MS (M+H)⁺=425

F1.4: 2-[[4-[4-(Dimethylamino)-1-piperidinyl]-6-[[(3,4,5-trimethoxyphenyl)methyl]amino]-2-pyrimidinyl]amino]-4-methyl-5-thiazolecarboxylic Acid, Ethyl Ester

A suspension of F1.4 (1.1 g, 2.6 mmol) and 3,4,5-trimethoxybenzylamine (1.12 ml, 5.7 mmol) in n-butanol (20 ml) was heated to 130° C. overnight. After cooling to room temperature, the precipitated solid was collected by filtration and washed with methanol to yield F1 (1.2 g, 80% ). ¹H-NMR (DMSO-d₆) δ: 6.68 (2H, s), 5.42 (1 H, s), 4.50-4.30 (2H, br. m), 4.13 (2H, q, J=7Hz), 3.69 (6H, s), 3.57 (3H, s), 2.82 (2H, m), 2.64 (3H, s), 2.63 (6H, s ), 2.20-2.11 (2H, m), 2.09-2.00 (3H, m), 1.93-1.78 (2H, m ), 1.58-1.46 (2H, m ), 1.19 (3H, t, J=7 Hz ). HPLC: 95%, ret. time=1.393 min., LC/MS (M+H)⁺=586.

F2: 2-[4,6-Bis-(4-hydroxy-piperidin-1-yl)-pyrimidin-2-ylamino]-4-methyl-thiazole-5-carboxylic Acid Ethyl Ester

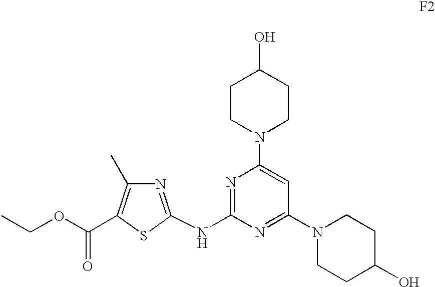

F1.3 (2.0 g, 6 mmol) and 4-hydroxypiperidine (2.5 g, 24 mmol) were added to n-butanol and heated in a bath at 130° C. overnight (18h). F2 (1.0 g, 65%) as a pale yellow solid was filtered from the reaction solution. ¹H-NMR ( DMSO-d₆) δ: 11.0, (1H, br,s) 5.63 (1H, s), 4.22 (2H, br s), 4.13 (2H, q, J=7 Hz), 4.05 (4H, br. m), 3.65 (2H, br m), 3.18 (4H, t, J=12 Hz), 2.54 (3H, s), 1.90-1.70(4H, m), 148-1.32 (4H, m), 1.30 (3H, t, J=7 Hz). HPLC: 95%, ret. time=1.45 min., LC/MS (M+H)⁺=463.01.

### Example G

### In Vitro Data

IC₅₀ determination for Example F2 for each of the reported PDE enzymes was performed as described in the description of the SPA assay for cAMP. The LPS human PBMC TNF production assay was performed as described above.

| | | | | | | | LPS |
|---|---|---|---|---|---|---|---|
| | | | | | PDE5 | PDE6 | PBMC |
| | PDE7 | PDE1 | PDE3 | PDE4 | IC₅₀ | IC₅₀ | TNF |
| Example | IC₅₀ μM | IC₅₀ μM | IC₅₀ μM | IC₅₀ μM | μM | μM | IC₅₀ μM |
| F1 | 0.030 | 4.3 | 32 | 3.0 | 2.8 | 7.5 | ND |
| F2 | 0.060 | 4.8 | 20 | 3.2 | 0.72 | 0.73 | >25 |
| rolipram | >10 | ND | ND | 0.74 | ND | ND | ND |
| cilomilast | >50 | 3.4 | >10 | 0.030 | >10 | ND | 0.43 |

ND = Not determined

As can be seen from the table above Example F1 is 100 fold selective for PDE7 over PDE4 and example F2 is greater than 50 fold selective for PDE7. The IC₅₀ for LPS PBMC TNF was >25 micromolar for example F2 while cilomilast was potent in this assay with an IC₅₀ of 0.43 μM.

### Example H

### Pharmacokinetic Data in Mice for Example F1 and Rolipram

Mice were administered 30 mg/kg IP of F1 and 45 minutes later were administered 10 mg of rolipram orally. The Cₘₐₓ data from this experiment is presented in the table below. It can be seen that the Cₘₐₓ for F1 are essentially unchanged by co-administration of rolipram, and the Cₘₐₓ of Rolipram was reduced by a factor of 3 by co-administration with F1. Also of note is the plasma concentration of F1 when administered at 30 mg/kg does not reach the PDE4 IC₅₀ of example F1.

| | Cmax, μM | Cmax, μM |
|---|---|---|
| Treatment | F1 | Rolipram |
| Rolipram | — | 0.29 |
| Example F1 | 2.2 | — |
| Example F1 + | 2.0 | 0.9 |
| Rolipram | | |

### Example I-1

### Effect of the PDE4 Inhibitor Rolipram and the PDE7 Inhibitor F1 on Lipopolysaccharide (LPS) Induced Tumor Necrosis Factor (TNF) Production in Mice

Mice were exposed to lipopolysaccharide to induce production of tumor necrosis factor as described by Cornwell, et. al. (Lipopolysaccharide, but not lethal infection, releases tumor necrosis factor in mice. Cornwell, R. D.; Golenbock, D. T.; Proctor, R. A. Adv.; Exp. Med. Biol. (1990), 256(Endotoxin), 585-8.). The mice were divided in to groups of eight animals each. All animals received an intraperitoneal (IP) injection of 50 μg/kg of LPS. The vehicle control group of animals received 0.2 mL of a vehicle of tween80 (5%), 95% ethanol (5%) and water 90%, sixty minutes prior to administration of LPS, and an IP injection of 0.2 mL of water fifteen minutes prior to administration of LPS. A group of mice (rolipram group) received a oral dose of 5 mg/kg of rolipram in a vehicle of tween80 (5%), 95% ethanol (5%) and water 90%, sixty minutes prior to administration of LPS and an IP injection of 0.2 mL of water fifteen minutes prior to administration of LPS. A group of mice (F1 group) were administered 0.2 mL of a vehicle of tween80 (5%), 95% ethanol (5%) and water 90%, sixty minutes prior to administration of LPS, and Example F1, at a dose of 7.5 mg/kg, IP in water, fifteen minutes prior to administration of LPS. A group of mice (Rolipram+F1 group) were administered rolipram at a dose of 5 mg/kg in of a vehicle of tween80 (5%), 95% ethanol (5%) and water 90%, sixty minutes prior to administration of LPS, and Example F1, at a dose of 7.5 mg/kg, IP in water, fifteen minutes prior to administration of LPS. A group of mice (dexamethasone group) were administered a 5 mg/kg dose of dexamethasone in a vehicle of tween80 (5%), 95% ethanol (5%) and water 90%, sixty minutes prior to administration of LPS, and 0.2 mL of water IP, fifteen minutes prior to administration of LPS. Compared to LPS-injected mice pretreated with vehicle, mice receiving Example F1 or rolipram alone had 52% and 54% reductions in serum TNF, respectively (each p<0.05 vs vehicle), as measured by a specific immunoassay. Mice treated with the combination of rolipram plus Example F1 showed an 89% reduction in serum TNF, which was significantly (p<0.05) less than mice receiving either compound alone. Mice treated with dexamethasone showed a 93% reduction in serum TNF.

### Example I-2

### Effect of the PDE4 Inhibitor Cilomilast and the PDE7 Inhibitor F2 on Lipopolysaccharide (LPS) Induced Tumor Necrosis Factor (TNF) Production in Mice

This experiment was conducted in a manner similar to that described for example I-1, except that the PDE4 inhibitor was changed from rolipram to cilomilast at a dose of 1 mg/kg, and the PDE7 inhibitor was changed from F1 to F2 (dose at 30 mg/kg). Compound F2 inhibited TNF production by 33.7% which was not statistically significant in this experiment. Cilomilast inhibited TNF production by 56% (p<0.05). The combination group which received both cilomilast 1 mg/kg and compound F2, had a decrease in TNF production of 72% (p<0.05 vs cilomilast alone). Finally the dexamethasone control inhibited TNF production 94%.

We claim:

1. A method of treating leukocyte activation-associated diseases in a warm-blooded animal comprising administering to said warm-blooded animal a leukocyte activation-associated disease treating effective amount of at least one dual PDE7-PDE4 inhibitor for which the IC₅₀ in both a PDE7 and a PDE4 inhibition assay is less than 20 micromolar, and the IC₅₀ in a PDE3 inhibition assay is at least 10 times higher than the IC₅₀ of the compound in the PDE7 assay.

2. The method of claim 1 wherein the dual PDE7-PDE4 inhibitor is a compound for which the IC₅₀ in both a PDE7 and a PDE4 inhibition assay is less than 5 micromolar, and the IC₅₀ in a PDE3 inhibition assay is at least 100 times higher than the IC₅₀ of the compound in the PDE7 assay.

3. The method of claim 1 wherein the dual PDE7-PDE4 inhibitor further inhibits PDE1 with an IC₅₀ at least 10 times higher than the IC₅₀ of the compound in a PDE7 assay.

4. The method of claim 1 wherein the dual PDE7-PDE4 inhibitor is a compound that suppresses both T cell proliferation and TNF-alpha production at a level of less than 20 micromolar.

5. The method of claim 1 wherein the leukocyte activation-associated disease is transplant rejection.

6. The method of claim 1 wherein the leukocyte activation-associated disease is rheumatoid arthritis.

7. The method of claim 1 wherein the leukocyte activation-associated disease is inflammatory bowel disease.

8. The method of claim 1 wherein the leukocyte activation-associated disease is psoriasis.

9. The method of claim 1 wherein the leukocyte activation-associated disease is asthma.

10. The method of claim 1 wherein the leukocyte activation-associated disease is lupus.

11. The method of claim 1 wherein the leukocyte activation-associated disease is COPD.

12. The method of claim 1 wherein the leukocyte activation-associated disease is multiple sclerosis.

13. The method of claim 1 wherein said dual PDE7-PDE4 inhibitor is administered in combination with at least one additional therapeutic agent suitable for treatment of leukocyte activation-associated diseases.

14. The method of claim 1 wherein said dual PDE7-PDE4 inhibitor is a compound of formula Ia or Ib 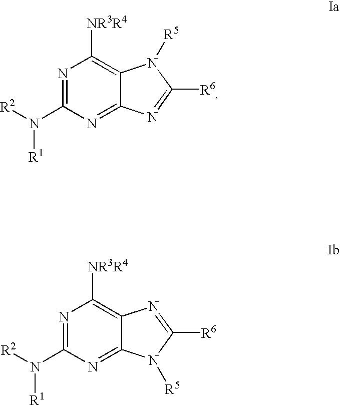
wherein
R¹ is H or alkyl;
R² is optionally substituted heteroaryl, or 4-substituted aryl;
R³ is hydrogen or alkyl;
R⁴ is alkyl, optionally substituted (aryl)alkyl, optionally substituted (heteroaryl)alkyl, optionally substituted heterocylo, or optionally substituted (heterocyclo)alkyl;
or R³ and R⁴ together with the nitrogen atom to which they are attached may combine to form an optionally substituted heterocyclo ring;
R⁵ is alkyl, optionally substituted (aryl)alkyl, or optionally substituted (heteroaryl)alkyl; and
R⁶ is hydrogen or alkyl.

15. The method of claim 1 wherein said dual PDE7-PDE4 inhibitor is a compound of formula II 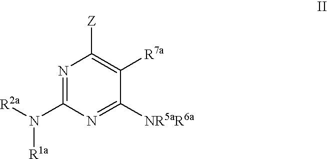
wherein
R^{1a} is H or alkyl;
R^{2a} is optionally substituted heteroaryl;
Z is halogen, alkyl, substituted alkyl, haloalkyl, or NR₃ₐR^{4a};
R^{3a} is hydrogen or alkyl;
R^{4a} is alkyl, optionally substituted (heteroaryl)alkyl, optionally substituted heterocylo, optionally substituted (heterocyclo)alkyl, or (aryl)alkyl wherein the aryl group is substituted with one or two groups T^{1*} and T^{2*} and optionally further substituted with a group T^{3*};
or R^{3a} and R^{4a} together with the nitrogen atom to which they are attached may combine to form an optionally substituted heterocyclo ring;
R^{5a} is (aryl)alkyl wherein the aryl group is substituted with one or two groups T^{1*} and T^{2*} and optionally further substituted with a group T^{3*};
R^{6a} is hydrogen or alkyl;
R^{7a} is hydrogen or alkyl;
T^{1*} and T^{2*} are independently alkoxy, alkoxycarbonyl, heteroaryl or —SO₂R^{8a} where R^{8a} is alkyl, amino, alkylamino or dialkylamino;
or T^{1*} and T^{2*} together with the atoms to which they are attached may combine to form a ring (e.g., benzodioxole);
T^{3*} is H, alkyl, halo, haloalkyl or cyano.

16. The method of claim 1 wherein said dual PDE7-PDE4 inhibitor is a compound of formula III. 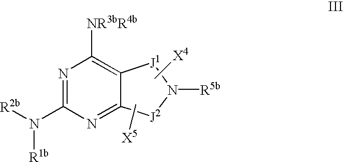
wherein
R^{1b} is H or alkyl;
R^{2b} is optionally substituted heteroaryl;
R^{3b} is H or alkyl;
R^{4b} is optionally substituted (aryl)alkyl;
R^{5b} is H, alkyl, or —C(O)—(CH₂)ᵥ—O—Y—R^{6b}, where Y is a bond or —C(O)—, R^{6b} is hydrogen or alkyl, and v is an integer from 0 to 2;
J¹ and J² are independently optionally substituted C₁₋₁₃ alkylene, provided that J¹ and J² are not both greater than C₂ alkylene;
X⁴ and X⁵ are optional substituents bonded to any available carbon atom in one or both of J¹ and J², independently selected from hydrogen, OR⁷, NR⁸R⁹, alkyl, substituted alkyl, alkenyl, substituted alkenyl, alkynyl, substituted alkynyl, cycloalkyl, substituted cycloalkyl, aryl, substituted aryl, heterocycloalkyl, or heteroaryl;
R⁷ is hydrogen, alkyl, substituted alkyl, alkenyl, alkynyl, cycloalkyl, substituted cycloalkyl, C(O)alkyl, C(O)substituted alkyl, C(O)cycloalkyl, C(O) substituted cycloalkyl, C(O)aryl, C(O)substituted aryl, C(O)Oalkyl, C(O)Osubstituted alkyl, C(O)heterocycloalkyl, C(O)heteroaryl, aryl, substituted aryl, heterocycloalkyl and heteroaryl; and
R⁸ and R⁹ are independently selected from the group consisting of hydrogen, alkyl, substituted alkyl, cycloalkyl, substituted cycloalkyl, alkenyl, alkynyl, C(O)alkyl, C(O)substituted alkyl, C(O)cycloalkyl, C(O)substituted cycloalkyl, C(O)aryl, C(O)substituted aryl, C(O)Oalkyl, C(O)Osubstituted alkyl, C(O)heterocycloalkyl, C(O)heteroaryl, S(O)₂alkyl, S(O)₂substituted alkyl, S(O)₂cycloalkyl, S(O)₂substituted cycloalkyl, S(O)₂aryl, S(O)₂substituted aryl, S(O)₂heterocycloalkyl, S(O)₂heteroaryl, aryl, substituted aryl, heterocycloalkyl, and heteroaryl, or R₈ and R₉ taken together with the nitrogen atom to which they are attached complete an optionally substituted heterocycloalkyl or heteroaryl ring.

17. The method of claim 1 wherein said dual PDE7-PDE4 inhibitor is a compound of formula IV. 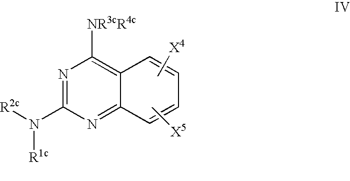
wherein
R^{1c} is H or alkyl;
R^{2c} is optionally substituted heteroaryl;
R^{3c} is H or alkyl;
R^{4c} is optionally substituted (aryl)alkyl; and
X⁴ and X⁵ are optional substituents bonded to any available carbon atom in one or both of J¹ and J², independently selected from hydrogen, OR⁷, NR⁸R⁹, alkyl, substituted alkyl, alkenyl, substituted alkenyl, alkynyl, substituted alkynyl, cycloalkyl, substituted cycloalkyl, aryl, substituted aryl, heterocycloalkyl, or heteroaryl.

18. A method of reducing emesis or nausea associated with the administration of PDE4 inhibitors for the treatment of leukocyte activation-associated disease comprising simultaneously or sequentially co-administering an effective amount of a selective PDE7 inhibitor together with and an effective lesser amount of said PDE4 inhibitor to a warm-blooded animal in need of such treatment.

19. The method of claim 18 wherein the PDE4 inhibitor is selected from Arofyline, Cilomilast, Roflumilast, C-11294A, CDC-801, BAY-19-8004, Cipamfylline, SCH351591, YM-976, PD-189659, Mesiopram, Pumafentrine. CDC-998, IC-485, and KW-4490.

20. A method of reducing emesis or nausea associated with the administration of PDE4 inhibitors for the treatment of leukocyte activation-associated disease comprising administering an effective amount of a dual PDE7-PDE4 inhibitor to a warm-blooded animal in need of such treatment.
